(19) United States
(12) Patent Application Publication
(10) Pub. No.: US 2025/0161304 A1
(43) Pub. Date: May 22, 2025

(54) Methods for Treating Cancer Using Pyrimidine and Pyridine Compounds with BTK Inhibitory Activity

(71) Applicant: Merck Patent GmbH, Darmstadt (DE)

(72) Inventors: Bayard R. Huck, Sudbury, MA (US); Samantha M. Goodstal, Lexington, MA (US); Claude Gimmi-McKim, Moehlin (CH)

(73) Assignee: Merck Patent GmbH, Darmstadt (DE)

(21) Appl. No.: 18/953,295

(22) Filed: Nov. 20, 2024

Related U.S. Application Data
(63) Continuation of application No. 17/232,383, filed on Apr. 16, 2021, now abandoned.
Application No. 17/232,383 is a continuation of application No. 15/343,570, filed on Nov. 4, 2016, now abandoned.
(60) Provisional application No. 62/341,189, filed on May 25, 2016.
(60) Provisional application No. 62/296,143, filed on Feb. 17, 2016.
(60) Provisional application No. 62/250,575, filed on Nov. 4, 2015.

(51) Int. Cl. A61K 31/506; A61K 31/505; A61K 31/5377; A61K 31/55; A61K 31/69; A61P 35/00; C07D 401/12

(52) U.S. Cl. CPC A61K 31/506; A61K 31/505; A61K 31/5377; A61K 31/55; A61K 31/69; A61P 35/00; C07D 401/12

(57) ABSTRACT

The present invention provides methods of treating cancer using pyrimidine and pyridine compounds which are inhibitors of Bruton's tyrosine kinase (BTK).

### CROSS-REFERENCE TO RELATED APPLICATIONS

This application is a continuation of U.S. patent application Ser. No. 17/232,383, filed on Apr. 16, 2021, which is a continuation of U.S. patent application Ser. No. 15/343,570, filed on Nov. 4, 2016, and claims the benefit of U.S. Provisional Application Nos. 62/250,575, filed on Nov. 4, 2015, 62/296,143, filed on Feb. 17, 2016, and 62/341, 189, filed on May 25, 2016. The content of each of these applications is hereby incorporated by reference in its entirety.

### BACKGROUND OF THE INVENTION

### Field of the invention

The invention relates to a series of pyrimidine and pyridine compounds that are useful as therapeutics in the treatment of cancer in mammals. More particularly, embodiments of the present invention describe irreversible kinase inhibitors including, but not limited to, inhibitors of Bruton's tyrosine kinase (hereinafter referred to as: “BTK”). Methods for the preparation of the aforementioned compounds are disclosed in addition to the incorporation of these compounds into pharmaceutical compositions that include the same.

### Description of Related Art

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, CA). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

BTK, a member of the Tec family of non-receptor tyrosine kinases, is a signaling enzyme expressed in all hematopoietic cells types except T lymphocytes and natural killer cells. BTK plays a well documented role in the B-cell signaling pathway linking cell surface B-cell receptor stimulation to downstream intracellular responses. BTK is also a regulator of B-cell development, activation, signaling, and survival (Kurosaki, Curr Op Imm, 2000, 276-281; Schaeffer and Schwartzberg, Curr Op Imm 2000, 282-288). In addition, BTK exerts a physiological effect through other hematopoetic cell signaling pathways, e.g.,

Toll like receptor (TLR) and cytokine receptor-mediated TNF-a production in macrophages, IgE receptor (FcepsilonRI) signaling in Mast cells, inhibition of Fas/APO-1 apoptotic signaling in B-lineage lymphoid cells, and collagen-stimulated platelet aggregation. BTK has an ATP-binding pocket with high similarity to Src-family kinases, such as lymphocyte-specific protein tyrosine kinase (Lck) and Lyn. Comparing BTK to other kinases one finds a conserved cysteine residue, Cys-481, in 11 of 491 kinases, specifically members of the Tec and EGFR (epidermal growth factor receptor) kinase families.

BTK plays important roles in the development, differentiation, activation and proliferation of B cells, as well as their antibody and cytokine generation. In addition, Btk plays a central role in other immunological processes such as cytokine production by neutrophils, mast cells and monocytes, degranulation of neutrophils and mast cells as well as differentiation/activation of osteoclasts. B-cell activation, break of tolerance and auto-antibody production, on one hand and the proinflammatory milieu originated from exacerbated activation of monocytes, neutrophils and mast cells, on the other hand, are crucial in the etiology of autoimmune diseases, including (but not limited to) rheumatoid arthritis and systemic lupus erythematosus.

Reversible kinase inhibitors have been developed into therapeutic compounds. These reversible inhibitors, however, have decided disadvantages. Many reversible inhibitors of kinases interact with the ATP-binding site. Given the structure of the ATP-binding sites are highly conserved among kinases, it has been difficult to develop a reversible inhibitor that selectively inhibits a desired (i.e., target) kinase. Moreover, given that many reversible kinase inhibitors readily dissociate from their target polypeptide(s), maintaining inhibition over extended periods of time can be difficult. When using reversible kinase inhibitors as therapeutics, therefore, often times near toxic dosages and/or frequent dosing is required to achieve the intended biological effect.

What is needed, therefore, are irreversible kinase inhibitors that covalently bind to their target polypeptide(s) without (substantially) binding to off-target polypeptides and, thereby, exerting undesirable off-target effects.

### SUMMARY OF THE INVENTION

The present invention is directed towards compounds of the formulae presented herein for the treatment and/or prophylaxis of cancer, including Non-Hodgkin's Lymphoma mantle cell lymphoma and Non-Hodgkins's Lymphoma diffuse large b-cell lymphoma, including abc subtype.

### BRIEF DESCRIPTION OF THE DRAWINGS

FIG. 1 shows the average percent inhibition of compound (2) at each dose level across 11 primary CLL patient samples.

FIG. 2 show inhibition of compound (2) in an ABC-DLBCL assay, across 6 cell lines.

FIG. 3 show inhibition of compound (2) in an MCL assay, across 6 cell lines.

FIG. 4a shows the In vivo efficacy study in MCL model. The objective was to determine anti-tumor effects and dose response. MCL cell lines: Mino, s.c. xenograft models. Mouse strain: nu/nu mice. Treatments start at 150-200 mm³ tumor volume.

FIG. 4b: Mino: BTK inhibitor in MCL model, tumor volume and body weight.

FIG. 4c: Mino: BTK inhibitor in MCL model, tumor volume and body weight.

FIG. 4d: Mino: BTK inhibitor in MCL model, tumor volume and body weight.

FIG. 4e: Mino: BTK inhibitor in MCL model, tumor volume and body weight.

### DETAILED DESCRIPTION OF THE INVENTION

The present invention provides a series of novel pyrimidine and pyridine kinase inhibitors. In some embodiments said kinase inhibitors are irreversible inhibitors of tyrosine kinases. In preferred embodiments, said irreversible kinase inhibitors inhibit

BTK. While it is not intended that the compounds described by the present invention be limited to any specific mechanism of action, in some embodiments said irreversible kinase inhibitors exert a physiological effect by forming a covalent bond with Cys 481 in BTK. Significantly, this Cys 481 in BTK finds homologs in other kinases. Embodiments of the present invention also described methods for synthesizing said irreversible inhibitors, methods for using said irreversible inhibitors in the treatment of diseases (including hyperproliferative diseases). Further described are pharmaceutical formulations that include an irreversible kinase inhibitor including pharmaceutically acceptable salts, solvates or prodrugs thereof, that are kinase inhibitors and useful in the treatment of the above mentioned diseases.

In one aspect, the invention provides a method for the treatment and/or prophylaxis of cancer, comprising administering to a subject a compound of Formula (I):

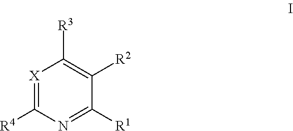

in which:
X denotes CH or N,
R¹ denotes NH₂, CONH₂ or H,
R² denotes Hal, Ar¹ or Het¹,
R³ denotes NR⁵[C(R⁵)₂]ₙHet², NR⁵[C(R⁵)₂]ₙCyc, Het², O[C(R⁵)₂]ₙAr², NR⁵[C(R⁵)₂]ₙAr², O[C(R⁵)₂]ₙHet², NR⁵(CH₂)ₚNR⁵R⁶, O(CH₂)ₚNR⁵R⁶ or NR⁵(CH₂)ₚCR⁷R⁸NR⁵R⁶,
R⁴ denotes H, CH₃ or NH₂,
R⁵ denotes H or alkyl having 1, 2, 3 or 4 C atoms,
R⁶ N(R⁵)₂CH₂CH═CHCONH, Het³CH₂CH═CHCONH, CH₂═CHCONH(CH₂)ₙ, Het⁴(CH₂)ₙCOHet³-diyl-CH₂CH═CHCONH, HC≡CCO, CH₃C≡CCO, CH₂═CH—CO, CH₂═C(CH₃)CONH, CH₃CH═CHCONH(CH₂)ₙ, NECCR⁷R⁸CONH(CH₂)ₙ, Het⁴NH(CH₂)ₚCOHet³-diyl-CH₂CH═CHCONH, Het⁴ (CH₂)ₚCONH(CH₂CH₂O)ₚ(CH₂)ₚCOHet³-diyl-CH₂CH═CHCONH, CH₂═CHSO₂, ACH═CHCO, CH₃CH═CHCO, Het⁴(CH₂)ₚCONH(CH₂)ₚHet³-diyl-CH₂CH═CHCONH, Ar³CH═CHSO₂, CH₂═CHSO₂NH or N(R⁵)CH₂CH═CHCO,
R⁷, R⁸ denote together alkylene having 2, 3, 4, or 5 C atoms,
Ar¹ denotes phenyl or naphthyl, each of which is unsubstituted or mono-, di- or trisubstituted by R⁶, Hal, (CH₂)ₙNH₂, CONHAr³, (CH₂)ₙNHCOA, O(CH₂)ₙAr³, OCyc, A, COHet³, OA and/or OHet³(CH₂),
Ar² denotes phenyl, naphthyl or pyridyl each of which is unsubstituted or mono-, di- or trisubstituted by R⁶, Hal, OAr³, (CH₂)ₙNH₂, (CH₂)ANHCOA and/or Het³,
Ar³ denotes phenyl, which is unsubstituted or mono-, di- or trisubstituted by OH, OA, Hal, CN and/or A,
Het¹ denotes a mono- or bicyclic saturated, unsaturated or aromatic heterocycle having 1 to 4 N, O and/or S atoms, which may be unsubstituted or mono-, di- or trisubstituted by R⁶, O(CH₂)ₙAR³ and/or (CH₂)ₙAR³,
Het² denotes a mono- or bicyclic saturated heterocycle having 1 to 4 N, O and/or S atoms, which may be unsubstituted or mono-, di- or trisubstituted by R⁶, Het³, CycSO₂, OH, Hal, COOH, OA, COA, COHet³, CycCO, SO₂ and/or ═O,
Het³ denotes a monocyclic unsaturated, saturated or aromatic heterocycle having 1 to 4 N, O and/or S atoms, which may be unsubstituted or mono-, di- or trisubstituted by Hal, A and/or ═O,
Het⁴ denotes a bi- or tricyclic unsaturated, saturated or aromatic heterocycle having 1 to 4 N, O and/or S atoms, which may be unsubstituted or mono-, di-, tri- or tetrasubstituted by A, NO₂, Hal and/or ═O,
Cyc denotes cyclic alkyl having 3, 4, 5 or 6 C atoms, which is un-substituted, monosubstituted or disubstituted by R⁶ and/or OH and which may comprise a double bond,
A denotes unbranched or branched alkyl having 1-10 C atoms, in which 1-7 H atoms may be replaced by F and/or Cl and/or in which one or two non-adjacent CH₂ and/or CH-groups may be replaced by O, NH and/or by N,
Hal denotes F, Cl, Br or I,
n denotes 0, 1, 2, 3 or 4,
p denotes 1, 2, 3, 4, 5 or 6,
   and pharmaceutically usable salts, tautomers and stereoisomers thereof, including mixtures thereof in all ratios.

In general, all residues which occur more than once may be identical or different, i.e. are independent of one another. In other embodiments, the residues and parameters have the meanings indicated for the Formula (I), unless expressly indicated otherwise.

In certain embodiments, Het¹ denotes piperidinyl, piperazinyl, pyrrolidinyl, morpholinyl, furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, benzimidazolyl, benzotriazolyl, indolyl, benzo-1,3-dioxolyl, indazolyl, azabicyclo [3.2.1]octyl, azabicyclo [2.2.2]octyl, imidazolidinyl, azetidinyl, azepanyl, benzo-2,1,3-thiadiazolyl, tetrahydrofuryl, dioxolanyl, tetrahydrothienyl, dihydropyrrolyl, tetrahydroimidazolyl, dihydropyrazolyl, tetrahydropyrazolyl, tetrahydropyridyl, dihydropyridyl or dihydrobenzodioxinyl, each of which is unsubstituted or mono-, di- or trisubstituted by R⁶, O (CH₂)ₙAR³ and/or (CH₂)ₙAR³.

In certain embodiments, Het¹ denotes pyrazolyl, pyridyl, pyrimidinyl, dihydropyridyl or dihydrobenzodioxinyl, each of which is unsubstituted or mono-, di- or trisubstituted by R⁶, O(CH₂)ₙAR³ and/or (CH₂)ₙAR³.

In certain embodiments, Het² denotes piperidinyl, piperazinyl, pyrrolidinyl, morpholinyl, azabicyclo [3.2.1]octyl, azabicyclo [2.2.2]octyl, 2,7-diazaspiro [3.5]nonyl, 2,8-diazaspiro [4.5]decyl, 2,7-diazaspiro [4.4]nonyl, 3-azabicylo [3.1.0]hexyl, 2-azaspiro [3.3]heptyl, 6-azaspiro [3.4]octyl, 7-azaspiro [3.5]nonyl, 5-azaspiro [3.5]nonyl, imidazolidinyl, azetidinyl, azepanyl, tetrahydrofuryl, dioxolanyl, tetrahydrothienyl, tetrahydroimidazolyl, tetrahydropyrazolyl, tetrahydropyridyl, each of which is unsubstituted or mono-, di- or trisubstituted by R⁶, Het³, CycSO₂, OH, OA, COA, COHet³, CycCO, SO₂ and/or ═O.

In certain embodiments, Het³ denotes piperidinyl, piperazinyl, pyrrolidinyl, morpholinyl, furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, imidazolidinyl, azetidinyl, azepanyl, tetrahydrofuryl, dioxolanyl, tetrahydrothienyl, dihydro-pyrrolyl, tetrahydroimidazolyl, dihydropyrazolyl, tetrahydropyrazolyl, tetrahydropyridyl or dihydropyridyl, each of which may be unsubstituted or mono-, di- or trisubstituted by Hal, A and/or ═O.

In certain embodiments, Het³ denotes piperidinyl, pyrrolidinyl, morpholinyl, furyl, thienyl, pyrrolyl, imidazolyl, pyrazolyl, pyridyl, pyrimidinyl, dihydropyrrolyl, dihydropyrazolyl or dihydropyridyl, each of which may be unsubstituted or mono-, di- or trisubstituted by Hal, A and/or ═O.

In certain embodiments, Het⁴ denotes hexahydrothieno[3,4-d] imidazolyl, benzo [c][1,2,5] oxadiazolyl or 5H-dipyrrolo[1,2-c:2′, 1′-f][1,3,2]diazaborinin-4-ium-uidyl, each of which may be unsubstituted or mono-, di-, tri- or tetrasubstituted by A, NO₂, Hal and/or ═O.

In certain embodiments,
X denotes CH or N,
R¹ denotes NH₂, CONH₂ or H,
R² denotes Hal, AR¹ or Het¹,
R³ denotes NR⁵[C(R⁵)₂]ₙHet², NR⁵[C(R⁵)₂]ₙCyc, Het², O[C(R⁵)₂]ₙAR², NR⁵[C(R⁵)₂]ₙAR², O[C(R⁵)₂]ₙHet², NR⁵(CH₂)ₚNR⁵R⁶, O(CH₂)ₚNR⁵R⁶ or NR⁵(CH₂)ₚCR⁷R⁸NR⁵R⁶,
R⁴ denotes H,
R⁵ denotes H or alkyl having 1, 2, 3 or 4 C atoms,
R⁶ N(R⁵)₂CH₂CH═CHCONH, Het³CH₂CH═CHCONH, CH₂═CHCONH(CH₂)ₙ, Het⁴(CH₂)ₙCOHet³-diyl-CH₂CH═CHCONH, HC═CCO, CH₃C═CCO, CH₂═CH—CO, CH₂═C(CH₃) CONH, CH₃CH═CHCONH(CH₂)ₙ, N═CCR⁷R⁸CONH(CH₂)ₙ, Het⁴NH(CH₂)ₚCOHet³-diyl-CH₂CH═CHCONH, Het⁴(CH₂)ₚCONH(CH₂CH₂O)ₚ(CH₂)ₚCOHet³-diyl-CH₂CH═CHCONH, CH₂═CHSO₂, ACH═CHCO, CH₃CH═CHCO, Het⁴(CH₂)ₚCONH(CH₂)ₚHet³-diyl-CH₂CH═CHCONH, AR³CH═CHSO₂, CH₂═CHSO₂NH or N(R⁵)CH₂CH═CHCO,
R⁷, R⁸ denote together alkylene having 2, 3, 4, or 5 C atoms,
AR¹ denotes phenyl or naphthyl, each of which is unsubstituted or mono-, di- or trisubstituted by R⁶, Hal, (CH₂)ₙNH₂, CONHAr³, (CH₂)ₙNHCOA, O(CH₂)ₙAR³, OCyc, A, COHet³, OA and/or OHet³(CH₂), denotes phenyl or naphthyl, each of which is unsubstituted or mono-, AR² di- or trisubstituted by R⁶, Hal, OAR³, (CH₂) NH₂, (CH₂)ₙNHCOA and/or Het³,
AR³ denotes phenyl, which is unsubstituted or mono-, di- or trisubstituted by OH, OA, Hal, CN and/or A,
Het denotes piperidinyl, piperazinyl, pyrrolidinyl, morpholinyl, furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, benzimidazolyl, benzotriazolyl, indolyl, benzo-1,3-dioxolyl, indazolyl, azabicyclo [3.2.1]octyl, aza-bicyclo [2.2.2]octyl, imidazolidinyl, azetidinyl, azepanyl, benzo-2,1,3-thiadiazolyl, tetrahydrofuryl, dioxolanyl, tetrahydrothienyl, dihydro-pyrrolyl, tetrahydroimidazolyl, dihydropyrazolyl, tetrahydropyrazolyl, tetrahydropyridyl, dihydropyridyl or dihydrobenzodioxinyl, each of which is unsubstituted or mono-, di- or trisubstituted by R⁶, O(CH₂)ₙAR³ and/or (CH₂)ₙAR³,
Het² denotes piperidinyl, piperazinyl, pyrrolidinyl, morpholinyl, azabicyclo[3.2.1]octyl, azabicyclo[2.2.2]octyl, 2,7-diazaspiro[3.5]nonyl, 2,8-diazaspiro[4.5]decyl, 2,7-diazaspiro[4.4]nonyl, 3-azabicylo[3.1.0]hexyl, 2-azaspiro[3.3]heptyl, 6-azaspiro[3.4]octyl, 7-azaspiro[3.5]nonyl, 5-azaspiro[3.5]nonyl, imidazolidinyl, azetidinyl, azepanyl, tetrahydrofuryl, dioxolanyl, tetrahydrothienyl, tetrahydroimidazolyl, tetrahydropyrazolyl, tetra-hydropyridyl, each of which is unsubstituted or mono-, di- or trisubstituted by R⁶, Het³, CycSO₂, OH, OA, COA, COHet³, CycCO, SO₂ and/or ═O,
Het³ denotes piperidinyl, piperazinyl, pyrrolidinyl, morpholinyl, furyl, thienyl, pyrrolyl, imidazolyl, pyrazolyl, oxazolyl, isoxazolyl, thiazolyl, isothiazolyl, pyridyl, pyrimidinyl, triazolyl, tetrazolyl, oxadiazolyl, thiadiazolyl, pyridazinyl, pyrazinyl, imidazolidinyl, azetidinyl, azepanyl, tetrahydrofuryl, dioxolanyl, tetrahydrothienyl, dihydropyrrolyl, tetrahydroimidazolyl, dihydropyrazolyl, tetrahydropyrazolyl, tetra-hydropyridyl or dihydropyridyl, each of which may be unsubstituted or mono-, di- or trisubstituted by Hal, A and/or ═O,
Het⁴ denotes hexahydrothieno[3,4-d]imidazolyl, benzo[c][1,2,5]oxadiazolyl or 5H-dipyrrolo[1,2-c:2′, 1′-f][1,3,2]diazaborinin-4-ium-uidyl, each of which may be unsubstituted or mono-, di-, tri- or tetrasubstituted by A, NO₂, Hal and/or ═O,
Cyc denotes cyclic alkyl having 3, 4, 5 or 6 C atoms, which is un-substituted or monosubstituted by R⁶ and which may comprise a double bond,
A denotes unbranched or branched alkyl having 1-10 C atoms, in which 1-7 H atoms may be replaced by F and/or Cl and/or in which one or two non-adjacent CH₂ and/or CH-groups may be replaced by O, NH and/or by N,
Hal denotes F, Cl, Br or I,
n denotes 0, 1, 2, 3 or 4,
p denotes 1, 2, 3, 4, 5 or 6.

In certain embodiments, the invention provides a method for the treatment and/or prophylaxis of cancer, comprising administering to a subject a compound of Formula (II):

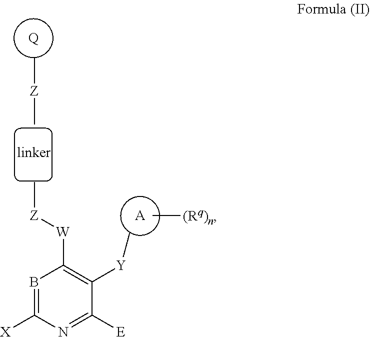

and pharmaceutically acceptable salts, solvates, solvates of salts, or prodrugs thereof, wherein:
X is H or CH₃ or NH₂,
Y is H, Hal or is absent,
B is N or CH,
E is NH₂ or H,
W is NR, O or a cyclic amine,
Z is, independently, CH₂, CH₃, CH₂-CH₂, CH—CH₂, H, NH or is absent,
“linker” is (CH₂)ₙ, wherein: n is 1, 2 or 3or an optionally substituted group selected from a phenyl ring, an aryl ring, heteroaryl ring, branched or unbranched alkyl group, a 5-6 membered monocyclic heteroaryl ring having 1-4 heteroatoms independently selected from nitrogen, or oxygen, a 4-7 membered saturated or partially unsaturated heterocycle having 1-3 heteroatoms independently selected from nitrogen, or oxygen, or a 7-10 membered bicyclic saturated or partially unsaturated heterocyclic ring having 1-5 heteroatoms independently selected from nitrogen, or oxygen, or a 7-10 membered bicyclic saturated or partially unsaturated heterocyclic ring having 1-5 heteroatoms attached to a hetero saturated ring. Linkers may also be cycloalkanes optionally substituted by heteroatoms (independently selected from nitrogen, or oxygen), cycloalkanes optionally substituted with —NH or OH, fused or bridged rings or optionally substituted spirocyclic rings that optionally contain heteroatoms,
A is a mono- or bicyclic aromatic homo- or heterocycle having 0, 1, 2, 3 or 4 N, and/or O atoms and 5, 6, 7, 8, 9, or 10 skeleton C atoms, which may be unsubstituted or, independently of one another, mono-, di- or trisubstituted by Hal, OH or OR,
Hal is F, Cl, Br or I,
R is independently hydrogen, oxygen or an optionally substituted group selected from C₁₋₆ linear or cyclic aliphatic, benzyl, phenyl, a phenyl group optionally substituted with 1, 2 or 3 O atoms, a 4-7 membered heterocylic ring having 1-2 heteroatoms independently selected from nitrogen, oxygen, or a 5-6 membered monocyclic heteroaryl ring having 1-4 heteroatoms independently selected from nitrogen, or oxygen or a mono- or bicyclic aromatic homo- or heterocycle having 0, 1, 2, 3 or 4 N, O atoms and 5, 6, 7, or 8 C skeleton atoms, which may be unsubstituted or, independently of one another, mono-, di- or trisubstituted by Hal, A, OH, NH₂, nitrile, and/or CH(Hal)₃ or is an unbranched or branched linear alkyl having 1, 2, 3, 4, 5, 6, 7 or 8 C atoms, in which one or two CH₂ groups may be replaced by an O atom and/or by an —NH—, —CO—, —NHCOO—, —NHCONH—, —CONH—, —NHCO— or —CH═CH-group, and in which 1-3 H atoms may be replaced by Hal,
R^{q} is selected from —R, —A, halogen, —OR, —O(CH₂)ᵣOR, —R(NH), —NO₂, —C(O)R, —CO₂R, —C(O)N(R)₂, —NRC(O)R, —NRC(O)NR², —NRSO₂R, or —N(R)₂,
r is 1-4,
n is 0-4, and
Q is an electrophilic group such as those listed in Table 1 wherein said electrophilic groups may further comprise a warhead.

As used herein the term “warhead” refers to a part, functional group or substituent of the compounds as claimed in the present invention, wherein, said part, functional group or substituent covalently binds to an amino acid (such as cysteine, lysine, or any other amino acid, either native or modified, that can form said covalent bond) that is present, for example, in the binding region within a given ligand wherein said warhead binds with said ligand, wherein the covalent binding between said warhead and the binding region of said target protein occurs under conditions wherein a physiological function of said protein is irreversibly inhibited.

While it is not intended that the present invention be limited to a specific group for substituent Q, as set out in Formula (II) above, in certain embodiments substituent Q is selected from the groups set out in Table 1. All compounds, in Table 1, appearing within a box are not “warheads” as defined above.

TABLE 1

| | | | |
|---|---|---|---|
| 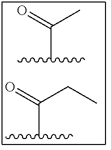 | 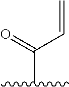 [SMILES: *C(=O)C=C] 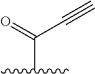 [SMILES: *C(=O)C#C] | 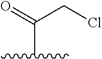 [SMILES: *C(=O)CCl] 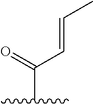 [SMILES: *C(=O)/C=C/C] | 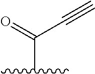 [SMILES: *C(=O)C#C] 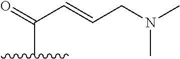 [SMILES: *C(=O)/C=C/CN(C)C] |
| 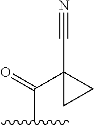 [SMILES: *C(=O)C1(C#N)CC1] | 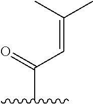 [SMILES: *C(=O)C=C(C)C] | 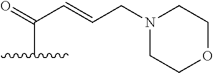 [SMILES: *C(=O)/C=C/CN1CCOCC1] | 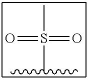 [SMILES: *S(C)(=O)=O] |
| 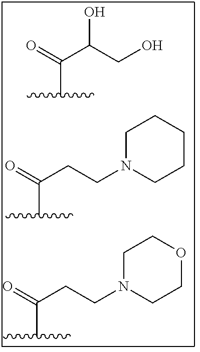 [SMILES: *C(=O)C(O)CO ; *C(=O)CCN1CCCCC1 ; *C(=O)CCN1CCOCC1] | 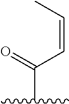 [SMILES: *C(=O)/C=C\C] 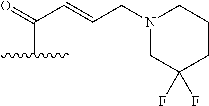 [SMILES: *C(=O)/C=C/CN1CCCC(F)(F)C1] | 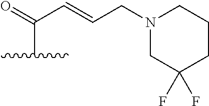 [SMILES: *C(=O)/C=C/CN1CCCC(F)(F)C1] 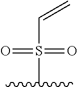 [SMILES: *S(=O)(=O)C=C] | 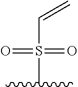 [SMILES: *S(=O)(=O)C=C] 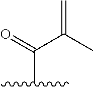 [SMILES: *C(=O)C(=C)C] |
| | 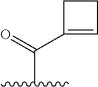 [SMILES: *C(=O)C1=CCC1] | 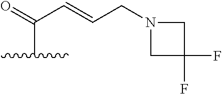 [SMILES: *C(=O)/C=C/CN1CC(F)(F)C1] | 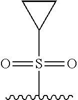 [SMILES: *S(=O)(=O)C1CC1] |
| 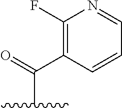 [SMILES: *C(=O)c1cccnc1F] | 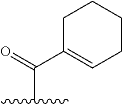 [SMILES: *C(=O)C1=CCCCC1] | 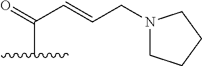 [SMILES: *C(=O)/C=C/CN1CCCC1] | 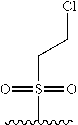 [SMILES: *S(=O)(=O)CCCl] |
| 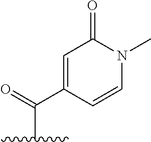 [SMILES: *C(=O)c1ccn(C)c(=O)c1] | 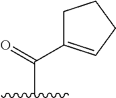 [SMILES: *C(=O)C1=CCCC1] | 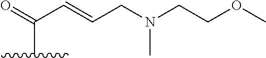 [SMILES: *C(=O)/C=C/CN(C)CCOC] | 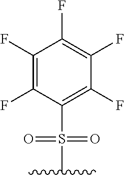 [SMILES: *S(=O)(=O)c1c(F)c(F)c(F)c(F)c1F] |

| | | |
|---|---|---|
| 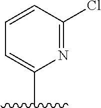 [SMILES: *c1cccc(Cl)n1] | 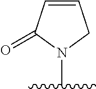 [SMILES: *N1CC=CC1=O] | 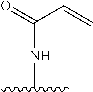 [SMILES: *NC(=O)C=C] |

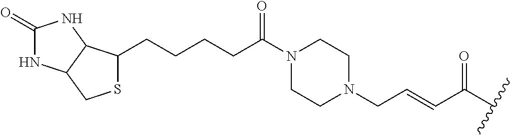

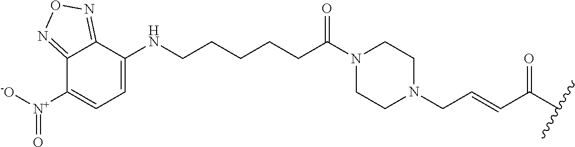

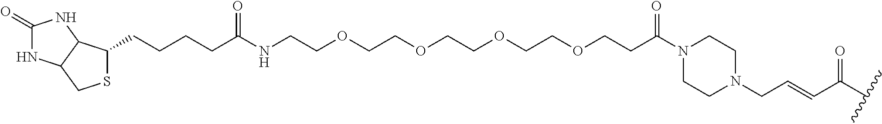

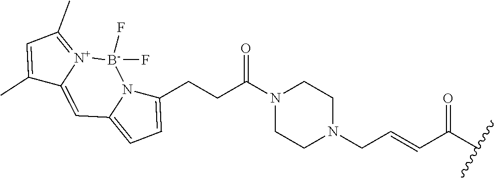

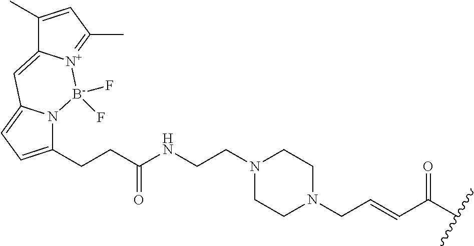

wherein, “ [symbol] ” denotes the bonding point of Q to Z in Formula (II).

In certain embodiments, the invention provides a method for the treatment and/or prophylaxis of cancer, comprising administering to a subject a compound of Formula (III):

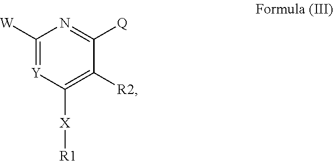

and pharmaceutically acceptable salts, solvates, solvates of salts, or prodrugs thereof, wherein:
X is O or NH,
Y is N or CH,
W is H, NH₂ or CONH₂,
Q is H or NH₂,
R¹ is L¹-R⁴-L²-R⁵,
R² is M¹-S⁴-M²-S⁵
L¹ is a single bond, methylene, or cyclic A which may be mono- or disubstituted with N or NH₂,
R⁴ is Ar, A or cyclic A which may be mono- or disubstituted with N, —O— or Hal,
R⁵ is Ar, A or cyclic A which may be mono- or disubstituted with N, —O— or Hal or is absent. In preferred embodiments, R⁵ is selected from the group consisting of 2-fluoropyridine, 1-methylpyridin-2 (1H)-one and 2-chloropyridine,
L² is H, —O—, substituted or unsubstituted C₁-C₄alkyl, substituted or unsubstituted C₁-C₄ heteroalkyl, C₁-C₆alkoxyalkyl, C₁-C₈alkylaminoalkyl, substituted or unsubstituted aryl, substituted or unsubstituted heteroaryl, C₁-C₄alkyl (aryl), C₁-C₄alkyl (heteroaryl), C₁-C₄alky (C₃-C₈cycloalkyl), or C₁-C₄alkyl (C₂-C₈heterocycloalkyl). In some embodiments, L² is —CH₂—O—(C₁-C₃alkyl), —CH₂—N(C₁-C₃alkyl)₂, C₁-C₄alkyl(phenyl), or C₁-C₄alkyl (5- or 6-membered heteroaryl). In some embodiments L² is —A—. In some embodiments L² is absent. In preferred embodiments of the present invention L² is selected from the group consisting of but-3-en-2-one, propan-2-one, (E)-5-(dimethylamino) pent-3-en-2-one, (E)-pent-3-en-2-one, pent-3-yn-2-one, 1-chloropropan-2-one, (methylsulfonyl) ethane, (E)-5-((2-methoxyethyl)(methyl)amino)pent-3-en-2-one or (Z)-pent-3-en-2-one,
M¹ is a single bond,
S⁴ is Ar, A or cyclic A which may be mono- or disubstituted with N, —O— or Hal. In preferred embodiments of the present invention S⁴ is a heteroaromatic 5 to 6 member ring,
M² O, NH, CH₂ or is absent,
S⁵ is H, Ar, A or cyclic A which may be mono- or disubstituted with N, —O—, Hal. In certain embodiments of the present invention S⁵ is selected from the group consisting of but-3-en-2-one, benzene, (E)-5-(dimethylamino)pent-3-en-2-one, ethylbenzene, 1-ethyl-2-methoxybenzene, aniline and (E)-5-morpholinopent-3-en-2-one. In some embodiments of the present invention S⁵ is absent,
Ar is a mono- or bicyclic aromatic homo- or heterocycle having 0, 1, 2, 3 or 4 N, and/or O atoms and 5, 6, 7, 8, 9, or 10 skeleton atoms, which may be unsubstituted or, independently of one another, mono-, di- or trisubstituted by Hal, A, OH, OA, NH₂, NHA, NA₂, NO₂, CN, OCN, COOH, COOA, CONH₂, CONHA, CONA₂, NHCOA, NHCONHA, NHCONH, CHO and/or COA, and in which a ring N-atom may be substituted by an O-atom to form an N-oxide group and in which in the case of a bicyclic aromatic cycle on of the two rings may be partly saturated,
A is unbranched or branched linear or cyclic alkyl having 1, 2, 3, 4, 5, 6, 7 or 8 C atoms, in which one or two CH₂ groups may be replaced by an O atom and/or by an —NH—, —CO—, —NHCOO—, —NHCONH—. —N(LA)—, —CONH—, —NHCO— or —CH═CH— group,

LA is unbranched or branched, linear alkyl having 1, 2, 3 or 4 C atoms, wherein 1, 2 or 3 H atoms may be replaced by Hal,

Hal is F, Cl, Br or I.

In certain embodiments, the invention provides a method for the treatment and/or prophylaxis of cancer, comprising administering to a subject a compound of Formula (IV):

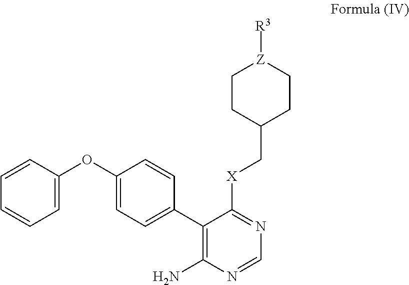

and pharmaceutically acceptable salts, solvates, solvates of salts, or prodrugs thereof, wherein:
Z is N or CH,
X is O or NH, and
R³ is selected from the group consisting of the following structures:

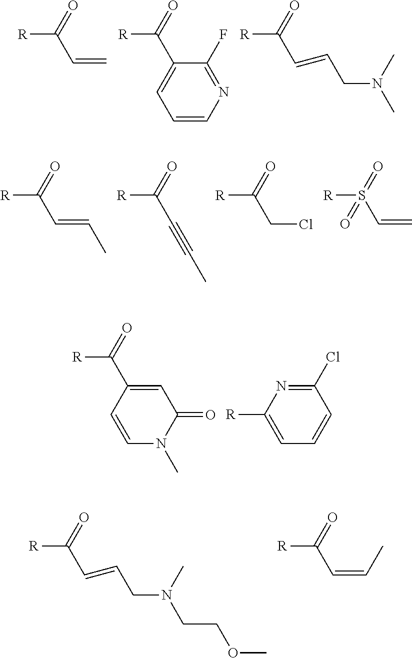

wherein, “R” denotes the bonding point to Z in Formula IV.

In certain embodiments, the invention provides a method for the treatment and/or prophylaxis of cancer, comprising administering to a subject a compound of Formula (V):

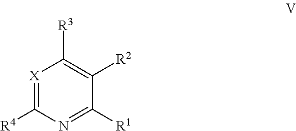

and pharmaceutically acceptable salts, solvates, solvates of salts, or prodrugs thereof, in which:
X denotes CH or N,
R¹ denotes NR⁵ [C (R⁵) 2]nHet²,
R² denotes Hal, AR¹ or Het¹,
R³ denotes NH₂,
R⁴ denotes H, CH₃ or NH₂,
R⁵ denotes H or alkyl having 1, 2, 3 or 4 C atoms,
R⁶ N(R⁵)₂CH₂CH═CHCONH, Het³CH₂CH═CHCONH, CH₂═CHCONH(CH₂)ₙ, Het⁴(CH₂)ₙCOHet³-diyl-CH₂CH═CHCONH, HC═CCO, CH₃C≡CCO, CH₂═CH—CO, CH₂═C(CH₃)CONH, CH₃CH═CHCONH(CH₂)ₙ, N═CCR⁷R⁸CONH(CH₂)ₙ, Het⁴NH (CH₂)ₚCOHet³-diyl-CH₂CH═CHCONH, Het⁴(CH₂)ₚCONH (CH₂CH₂O)ₚ(CH₂)ₚCOHet³-diyl-CH₂CH═CHCONH, CH₂═CHSO₂, ACH═CHCO, CH₃CH═CHCO, Het⁴(CH₂)ₚCONH(CH₂)ₚHet³-diyl-CH₂CH═CHCONH, AR³CH═CHSO₂, CH₂═CHSO₂NH or N(R⁵)CH₂CH═CHCO,
R⁷, R⁸ denote together alkylene having 2, 3, 4, or 5 C atoms,
AR¹ denotes phenyl or naphthyl, each of which is unsubstituted or mono-, di- or trisubstituted by R⁶, Hal, (CH₂)ₙNH₂, CONHAR³, (CH₂)ₙNHCOA, O(CH₂)ₙAR³, OCyc, A, COHet³, OA and/or OHet³(CH₂),
AR² denotes phenyl, naphthyl or pyridyl each of which is unsubstituted or mono-, di- or trisubstituted by R⁶, Hal, OAR³, (CH₂)ₙNH₂, (CH₂)ₙNHCOA and/or Het³,
AR³ denotes phenyl, which is unsubstituted or mono-, di- or trisubstituted by OH, OA, Hal, CN and/or A,
Het¹ denotes a mono- or bicyclic saturated, unsaturated or aromatic heterocycle having 1 to 4 N, O and/or S atoms, which may be unsubstituted or mono-, di- or trisubstituted by R⁶, O(CH₂)ₙAR³ and/or (CH₂)ₙAR³,
Het² denotes a mono- or bicyclic saturated heterocycle having 1 to 4 N, O and/or S atoms, which may be unsubstituted or mono-, di- or trisubstituted by R⁶, Het³, CycSO₂, OH, Hal, COOH, OA, COA, COHet³, CycCO, SO₂ and/or ═O,
Het³ denotes a monocyclic unsaturated, saturated or aromatic heterocycle having 1 to 4 N, O and/or S atoms, which may be unsubstituted or mono-, di- or trisubstituted by Hal, A and/or ═O,
Het⁴ denotes a bi- or tricyclic unsaturated, saturated or aromatic heterocycle having 1 to 4 N, O and/or S atoms, which may be unsubstituted or mono-, di-, tri- or tetrasubstituted by A, NO₂, Hal and/or ═O,
Cyc denotes cyclic alkyl having 3, 4, 5 or 6 C atoms, which is un-substituted, monosubstituted or disubstituted by R⁶ and/or OH and which may comprise a double bond,
A denotes unbranched or branched alkyl having 1-10 C atoms, in which 1-7 H atoms may be replaced by F and/or Cl and/or in which one or two non-adjacent CH₂ and/or CH-groups may be replaced by O, NH and/or by N,
Hal denotes F, Cl, Br or I,
n denotes 0, 1, 2, 3 or 4,
p denotes 1, 2, 3, 4, 5 or 6,
   and pharmaceutically usable salts, tautomers and stereoisomers thereof, including mixtures thereof in all ratios.

In certain embodiments, the invention provides a method for the treatment and/or prophylaxis of cancer, comprising administering to a subject a compound selected from Table 2:

TABLE 2

| No. | Chemical Name |
|---|---|
| “A1” | (R)-1-(3-((6-amino-5-(4-phenoxyphenyl)pyrimidin-4- |
| | yl)amino)pyrrolidin-1-yl)prop-2-en-1-one |
| “A2” | (R)-1-(3-((6-amino-5-(4-phenoxyphenyl)pyrimidin-4- |
| | yl)oxy)piperidin-1-yl)prop-2-en-1-one |
| “A3” | N-(3-((6-amino-5-(4-phenoxyphenyl)pyrimidin-4- |
| | yl)amino)phenyl)acrylamide |
| “A4” | (R)-1-(3-(((6-amino-5-(4-phenoxyphenyl)pyrimidin-4- |
| | yl)amino)methyl)pyrrolidin-1-yl)prop-2-en-1-one |
| “A5” | N-((1-(6-amino-5-(4-phenoxyphenyl)pyrimidin-4-yl)pyrrolidin-3- |
| | yl)methyl)acrylamide |
| “A6” | 1-(4-(((5-(4-phenoxyphenyl)pyrimidin-4-yl)oxy)methyl)piperidin- |
| | 1-yl)prop-2-en-1-one |
| “A7” | N-((1-(6-amino-5-(4-phenoxyphenyl)pyrimidin-4-yl)piperidin-4- |
| | yl)methyl)acrylamide |
| “A8” | 4-(4-(((6-amino-5-(4-phenoxyphenyl)pyrimidin-4- |
| | yl)amino)methyl)piperidine-1-carbonyl)-1-methylpyridin-2(1H)-one |
| “A9” | 1-(4-(((6-amino-5-(4-phenoxyphenyl)pyrimidin-4- |
| | yl)oxy)methyl)piperidin-1-yl)but-2-yn-1-one |
| “A10” | 5-(4-phenoxyphenyl)-N4-((1-(vinylsulfonyl)piperidin-4- |
| | yl)methyl)pyrimidine-4,6-diamine |
| “A11” | (E)-1-(4-(((6-amino-5-(4-phenoxyphenyl)pyrimidin-4- |
| | yl)amino)methyl)piperidin-1-yl)-4-((2- |
| | methoxyethyl)(methyl)amino)but-2-en-1-one |
| “A12” | (4-(((6-amino-5-(4-phenoxyphenyl)pyrimidin-4- |
| | yl)amino)methyl)piperidin-1-yl)(2-fluoropyridin-3-yl)methanone |
| “A13” | (E)-1-(4-(((6-amino-5-(4-phenoxyphenyl)pyrimidin-4- |
| | yl)amino)methyl)piperidin-1-yl)but-2-en-1-one |
| “A14” | N4-((1-(cyclopropylsulfonyl)piperidin-4-yl)methyl)-5-(4- |
| | phenoxyphenyl)pyrimidine-4,6-diamine |
| “A15” | (Z)-1-(4-(((6-amino-5-(4-phenoxyphenyl)pyrimidin-4- |
| | yl)amino)methyl)piperidin-1-yl)but-2-en-1-one |
| “A16” | 1-(4-(2-((6-amino-5-(4-phenoxyphenyl)pyrimidin-4- |
| | yl)amino)ethyl)piperidin-1-yl)prop-2-en-1-one |
| “A17” | 1-(3-(((6-amino-5-(4-phenoxyphenyl)pyrimidin-4- |
| | yl)oxy)methyl)piperidin-1-yl)prop-2-en-1-one |
| “A18” | N-(2-((6-amino-5-(4-phenoxyphenyl)pyrimidin-4- |
| | yl)amino)ethyl)acrylamide |
| “A19” | (R)-1-(3-((6-amino-5-(4-phenoxyphenyl)pyrimidin-4- |
| | yl)oxy)pyrrolidin-1-yl)prop-2-en-1-one |
| “A20” | N-(1-(((6-amino-5-(4-phenoxyphenyl)pyrimidin-4- |
| | yl)amino)methyl)cyclopentyl)acrylamide |
| “A21” | 1-(3-(((6-amino-5-(4-phenoxyphenyl)pyrimidin-4- |
| | yl)oxy)methyl)pyrrolidin-1-yl)prop-2-en-1-one |
| “A22” | 1-(4-(((5-fluoro-3-(4-phenoxyphenyl)pyridin-2- |
| | yl)amino)methyl)piperidin-1-yl)prop-2-en-1-one |
| “A23” | 1-(4-(((6-amino-5-(4-phenoxyphenyl)pyrimidin-4- |
| | yl)amino)methyl)piperidin-1-yl)ethanone |
| “A24” | (E)-7-(3-(4-(4-((3-((6-amino-5-(4-phenoxyphenyl)pyrimidin-4- |
| | yl)oxy)phenyl)amino)-4-oxobut-2-en-1-yl)piperazin-1-yl)-3- |
| | oxopropyl)-5,5-difluoro-1,3-dimethyl-5H-dipyrrolo[1,2-c: 2′,1′-f] |
| | [1,3,2]diazaborinin-4-ium-5-uide |
| “A25” | 1-(4-(((2-amino-5-(4-phenoxyphenyl)pyrimidin-4- |
| | yl)amino)methyl)piperidin-1-yl)prop-2-en-1-one |
| “A26” | (S)-1-(3-(((6-amino-5-(4-phenoxyphenyl)pyrimidin-4- |
| | yl)oxy)methyl)pyrrolidin-1-yl)prop-2-en-1-one |
| “A27” | N-(2-((6-amino-5-(4-phenoxyphenyl)pyrimidin-4- |
| | yl)oxy)ethyl)acrylamide |
| “A28” | (S)-1-(3-(((6-amino-5-(4-phenoxyphenyl)pyrimidin-4- |
| | yl)amino)methyl)pyrrolidin-1-yl)prop-2-en-1-one |
| “A29” | 1-(4-(((6-amino-5-(4-phenoxyphenyl)pyrimidin-4- |
| | yl)amino)methyl)piperidin-1-yl)-2-methylprop-2-en-1-one |
| “A30” | (4-(((6-amino-5-(4-phenoxyphenyl)pyrimidin-4- |
| | yl)amino)methyl)piperidin-1-yl)(cyclohex-1-en-1-yl)methanone |
| “A31” | 1-(4-(((6-amino-5-(4-phenoxyphenyl)pyrimidin-4- |
| | yl)amino)methyl)piperidin-1-yl)-3-methylbut-2-en-1-one |
| “A32” | (4-(((6-amino-5-(4-phenoxyphenyl)pyrimidin-4- |
| | yl)amino)methyl)piperidin-1-yl)(cyclopent-1-en-1-yl)methanone |
| “A33” | 1-(4-(((6-amino-5-(1-benzyl-1H-pyrazol-4-yl)pyrimidin-4- |
| | yl)amino)methyl)piperidin-1-yl)prop-2-en-1-one |
| “A34” | 1-(4-(((6-amino-5-(4-(3-fluorophenoxy)phenyl)pyrimidin-4- |
| | yl)amino)methyl)piperidin-1-yl)prop-2-en-1-one |
| “A35” | (E)-7-(3-((2-(4-(4-((3-((6-amino-5-(4-phenoxyphenyl)pyrimidin-4- |
| | yl)oxy)phenyl)amino)-4-oxobut-2-en-1-yl)piperazin-1- |
| | yl)ethyl)amino)-3-oxopropyl)-5,5-difluoro-1,3-dimethyl-5H- |
| | dipyrrolo[1,2-c: 2′,1′-f][1,3,2]diazaborinin-4-ium-5-uide |
| “A36” | 1-(4-(((6-amino-2-methyl-5-(4-phenoxyphenyl)pyrimidin-4- |
| | yl)amino)methyl)piperidin-1-yl)prop-2-en-1-one |
| “A37” | 1-(4-(((6-amino-5-(4-phenoxyphenyl)pyrimidin-4- |
| | yl)amino)methyl)-4-hydroxypiperidin-1-yl)prop-2-en-1-one |
| “A38” | (R)-1-(3-(((6-amino-5-(4-phenoxyphenyl)pyrimidin-4- |
| | yl)oxy)methyl)pyrrolidin-1-yl)prop-2-en-1-one |
| “A39” | 1-(4-(((6-amino-5-(4-(phenylamino)phenyl)pyrimidin-4- |
| | yl)amino)methyl)piperidin-1-yl)prop-2-en-1-one |
| “A40” | 1-(3-((6-amino-5-(4-phenoxyphenyl)pyrimidin-4- |
| | yl)oxy)phenyl)-1H-pyrrol-2(5H)-one |
| “A41” | 1-(4-(((6-amino-5-(4-benzylphenyl)pyrimidin-4- |
| | yl)amino)methyl)piperidin-1-yl)prop-2-en-1-one |
| “A42” | (4-(((6-amino-5-(4-phenoxyphenyl)pyrimidin-4- |
| | yl)amino)methyl)piperidin-1-yl)(cyclobut-1-en-1-yl)methanone |
| “A43” | (Z)-1-(4-(((6-amino-5-(4-phenoxyphenyl)pyrimidin-4- |
| | yl)oxy)methyl)piperidin-1-yl)but-2-en-1-one |
| “A44” | 1-(4-(((6-amino-2-methyl-5-(4-phenoxyphenyl)pyrimidin-4- |
| | yl)(methyl)amino)methyl)piperidin-1-yl)prop-2-en-1-one |
| “A45” | 1-(4-(((6-amino-5-(4-phenoxyphenyl)pyrimidin-4- |
| | yl)amino)methyl)piperidin-1-yl)-2-chloroethanone |
| “A46” | 1-(4-(((6-amino-5-(4-phenoxyphenyl)pyrimidin-4- |
| | yl)amino)methyl)piperidin-1-yl)prop-2-yn-1-one |
| “A47” | 1-(4-(((6-amino-5-(4-phenoxyphenyl)pyrimidin-4- |
| | yl)(methyl)amino)methyl)piperidin-1-yl)prop-2-en-1-one |
| “A48” | 1-(3-(((6-amino-5-(4-phenoxyphenyl)pyrimidin-4- |
| | yl)amino)methyl)-8-azabicyclo[3.2.1]octan-8-yl)prop-2-en-1-one |
| “A49” | N-((1S,3S)-3-((6-amino-5-(4-phenoxyphenyl)pyrimidin-4- |
| | yl)amino)cyclopentyl)acrylamide |
| “A50” | N-(4-((6-amino-5-(4-phenoxyphenyl)pyrimidin-4- |
| | yl)amino)butyl)acrylamide |
| “A51” | N-(cis-3-((6-amino-5-(4-phenoxyphenyl)pyrimidin-4- |
| | yl)amino)cyclohexyl)acrylamide |
| “A52” | 1-(3-((6-amino-5-(4-phenoxyphenyl)pyrimidin-4- |
| | yl)amino)azepan-1-yl)prop-2-en-1-one |
| “A53” | N-(trans-3-((6-amino-5-(4-phenoxyphenyl)pyrimidin-4- |
| | yl)amino)cyclohexyl)acrylamide |
| “A54” | (E)-5-(4-phenoxyphenyl)-N4-((1-(styrylsulfonyl)piperidin-4- |
| | yl)methyl)pyrimidine-4,6-diamine |
| “A55” | N4-((1-(methylsulfonyl)piperidin-4-yl)methyl)-5-(4- |
| | phenoxyphenyl)pyrimidine-4,6-diamine |
| “A56” | 1-(4-(((6-amino-5-(4-phenoxyphenyl)pyrimidin-4- |
| | yl)amino)methyl)piperidin-1-yl)-2,3-dihydroxypropan-1-one |
| “A57” | 4-(((6-amino-5-(4-phenoxyphenyl)pyrimidin-4- |
| | yl)amino)methyl)piperidin-2-one |
| “A58” | N-(3-((6-amino-5-(4-phenoxyphenyl)pyrimidin-4- |
| | yl)oxy)phenyl)ethenesulfonamide |
| “A59” | N-(3-((6-amino-5-(4-phenoxyphenyl)pyrimidin-4- |
| | yl)amino)propyl)acrylamide |
| “A60” | N-(5-((6-amino-5-(4-phenoxyphenyl)pyrimidin-4-yl)oxy)pyridin-3- |
| | yl)acrylamide |
| “A61” | (R)-1-(3-(((6-amino-5-(4-phenoxyphenyl)pyrimidin-4- |
| | yl)amino)methyl)pyrrolidin-1-yl)prop-2-yn-1-one |
| “A62” | (R,E)-1-(3-(((6-amino-5-(4-phenoxyphenyl)pyrimidin-4- |
| | yl)amino)methyl)pyrrolidin-1-yl)-4-(dimethylamino)but-2-en-1-one |
| “A63” | (E)-N-(cis-3-((6-amino-5-(4-phenoxyphenyl)pyrimidin-4- |
| | yl)amino)cyclohexyl)-4-(dimethylamino)but-2-enamide |
| “A64” | N-(cis-3-((6-amino-5-(4-phenoxyphenyl)pyrimidin-4- |
| | yl)amino)cyclohexyl)propiolamide |
| “A65” | (S)-1-(2-(((6-amino-5-(4-phenoxyphenyl)pyrimidin-4- |
| | yl)oxy)methyl)morpholino)prop-2-en-1-one |
| “A66” | (R)-1-(2-(((6-amino-5-(4-phenoxyphenyl)pyrimidin-4- |
| | yl)oxy)methyl)morpholino)prop-2-en-1-one |
| “A67” | N-(3-((6-amino-5-(1-(3-fluorobenzyl)-1H-pyrazol-4-yl)pyrimidin-4- |
| | yl)oxy)phenyl)acrylamide |
| “A68” | 1-(3-(((6-amino-5-(4-phenoxyphenyl)pyrimidin-4- |
| | yl)amino)methyl)-8-azabicyclo[3.2.1]octan-8-yl)prop-2-yn-1-one |
| “A69” | N-(3-((6-amino-5-(1-(4-cyanobenzyl)-1H-pyrazol-4-yl)pyrimidin- |
| | 4-yl)oxy)phenyl)acrylamide |
| “A70” | N-(3-((6-amino-5-(1-benzyl-1H-pyrazol-4-yl)pyrimidin-4- |
| | yl)amino)phenyl)acrylamide |
| “A71” | (E)-1-(3-(((6-amino-5-(4-phenoxyphenyl)pyrimidin-4- |
| | yl)amino)methyl)-8-azabicyclo[3.2.1]octan-8-yl)-4- |
| | (dimethylamino)but-2-en-1-one |
| “A72” | N-(3-((6-amino-5-(1-(4-methoxybenzyl)-1H-pyrazol-4- |
| | yl)pyrimidin-4-yl)oxy)phenyl)acrylamide |
| “A73” | (R,E)-1-(3-((6-amino-5-(4-phenoxyphenyl)pyrimidin-4- |
| | yl)amino)pyrrolidin-1-yl)-4-(dimethylamino)but-2-en-1-one |
| “A74” | (R,E)-1-(3-((6-amino-5-(4-phenoxyphenyl)pyrimidin-4- |
| | yl)amino)piperidin-1-yl)-4-(dimethylamino)but-2-en-1-one |
| “A75” | 1-(trans-3-((6-amino-5-(4-phenoxyphenyl)pyrimidin-4-yl)amino)- |
| | 4-hydroxypyrrolidin-1-yl)prop-2-en-1-one |
| “A76” | 1-(4-(((2-amino-3-(4-phenoxyphenyl)pyridin-4- |
| | yl)amino)methyl)piperidin-1-yl)prop-2-en-1-one |
| “A77” | 1-(4-(((6-amino-5-(4-fluorophenyl)pyrimidin-4- |
| | yl)amino)methyl)piperidin-1-yl)prop-2-en-1-one |
| “A78” | 1-(4-(((6-amino-5-(4-(trifluoromethoxy)phenyl)pyrimidin-4- |
| | yl)amino)methyl)piperidin-1-yl)prop-2-en-1-one |
| “A79” | 1-(4-(((6-amino-5-(4-(4- |
| | (trifluoromethyl)phenoxy)phenyl)pyrimidin-4- |
| | yl)amino)methyl)piperidin-1-yl)prop-2-en-1-one |
| “A80” | 1-(4-(((6-amino-5-(4-(4-(fluorophenoxy)phenyl)pyrimidin-4- |
| | yl)amino)methyl)piperidin-1-yl)prop-2-en-1-one |
| “A81” | 1-(4-(((6-amino-5-(4-(trifluoromethyl)phenyl)pyrimidin-4- |
| | yl)amino)methyl)piperidin-1-yl)prop-2-en-1-one |
| “A82” | 1-(4-(((6-amino-5-(3,4-dimethoxyphenyl)pyrimidin-4- |
| | yl)amino)methyl)piperidin-1-yl)prop-2-en-1-one |
| “A83” | 1-(4-(((6-amino-5-(3,4,5-trimethoxyphenyl)pyrimidin-4- |
| | yl)amino)methyl)piperidin-1-yl)prop-2-en-1-one |
| “A84” | 1-(4-(((6-amino-5-(2,3-dihydrobenzo[b][1,4]dioxin-6-yl)pyrimidin- |
| | 4-yl)amino)methyl)piperidin-1-yl)prop-2-en-1-one |
| “A85” | 1-(4-(((6-amino-5-(4-methoxyphenyl)pyrimidin-4- |
| | yl)amino)methyl)piperidin-1-yl)prop-2-en-1-one |
| “A86” | 4-(4-(4-(((1-acryloylpiperidin-4-yl)methyl)amino)-6- |
| | aminopyrimidin-5-yl)phenoxy)benzonitrile |
| “A87” | 1-(4-(((6-amino-5-(2,5-difluoro-4-phenoxyphenyl)pyrimidin-4- |
| | yl)amino)methyl)piperidin-1-yl)prop-2-en-1-one |
| “A88” | 1-(4-(((6-amino-5-(2,3-difluoro-4-phenoxyphenyl)pyrimidin-4- |
| | yl)amino)methyl)piperidin-1-yl)prop-2-en-1-one |
| “A89” | 1-(4-(((6-amino-5-(4-((1-methylpiperidin-4- |
| | yl)oxy)phenyl)pyrimidin-4-yl)amino)methyl)piperidin-1-yl)prop-2- |
| | en-1-one |
| “A90” | 1-(4-(((6-amino-5-(4-phenoxy-2- |
| | (trifluoromethyl)phenyl)pyrimidin-4-yl)amino)methyl)piperidin-1- |
| | yl)prop-2-en-1-one |
| “A91” | 1-(2-(6-amino-5-(4-phenoxyphenyl)pyrimidin-4-yl)-2,7- |
| | diazaspiro[3.5]nonan-7-yl)prop-2-en-1-one |
| “A92” | 1-(8-(6-amino-5-(4-phenoxyphenyl)pyrimidin-4-yl)-2,8- |
| | diazaspiro[4.5]decan-2-yl)prop-2-en-1-one |
| “A93” | 1-(7-(6-amino-5-(4-phenoxyphenyl)pyrimidin-4-yl)-2,7- |
| | diazaspiro[4.4]nonan-2-yl)prop-2-en-1-one |
| “A94” | 1-(4-(((6-amino-5-(4-(4-hydroxyphenoxy)phenyl)pyrimidin-4- |
| | yl)amino)methyl)piperidin-1-yl)prop-2-en-1-one |
| “A95” | 1-(4-(((6-amino-5-(4-(3- |
| | (trifluoromethyl)phenoxy)phenyl)pyrimidin-4- |
| | yl)amino)methyl)piperidin-1-yl)prop-2-en-1-one |
| “A96” | 1-(4-(((6-amino-5-(4-(pyridin-3-yloxy)phenyl)pyrimidin-4- |
| | yl)amino)methyl)piperidin-1-yl)prop-2-en-1-one |
| “A97” | 1-(4-(((6-amino-5-(4-(pyridin-4-yloxy)phenyl)pyrimidin-4- |
| | yl)amino)methyl)piperidin-1-yl)prop-2-en-1-one |
| “A98” | 1-(4-(((6-amino-5-(4-(p-tolyloxy)phenyl)pyrimidin-4- |
| | yl)amino)methyl)piperidin-1-yl)prop-2-en-1-one |
| “A99” | 1-(4-(((6-amino-5-(4-(cyclohexyloxy)phenyl)pyrimidin-4- |
| | yl)amino)methyl)piperidin-1-yl)prop-2-en-1-one |
| “A100” | N4-((1R,5S,6r)-3-azabicyclo[3.1.0]hexan-6-ylmethyl)-5-(4- |
| | phenoxyphenyl)pyrimidine-4,6-diamine hydrochloride |
| “A101” | (3S,4S)-4-(((6-amino-5-(4-phenoxyphenyl)pyrimidin-4- |
| | yl)amino)methyl)piperidin-3-ol hydrochloride |
| “A102” | (E)-1-(6-((6-amino-5-chloropyrimidin-4-yl)oxy)-2- |
| | azaspiro[3.3]heptan-2-yl)-4-(dimethylamino)but-2-en-1-one |
| “A103” | 1-(3-(2-((6-amino-5-(4-phenoxyphenyl)pyrimidin-4- |
| | yl)amino)ethyl)azetidin-1-yl)prop-2-en-1-one |
| “A104” | 1-(3-(2-((6-amino-5-(4-phenoxyphenyl)pyrimidin-4- |
| | yl)amino)ethyl)azetidin-1-yl)prop-2-yn-1-one |
| “A105” | (E)-1-(6-((6-amino-5-(4-phenoxyphenyl)pyrimidin-4-yl)oxy)-2- |
| | azaspiro[3.3]heptan-2-yl)-4-(dimethylamino)but-2-en-1-one |
| “A106” | 1-(6-((6-amino-5-(1-benzyl-1H-pyrazol-4-yl)pyrimidin-4-yl)oxy)-2- |
| | azaspiro[3.3]heptan-2-yl)prop-2-en-1-one |
| “A107” | 1-(6-((6-amino-5-(1-benzyl-1H-pyrazol-4-yl)pyrimidin-4- |
| | yl)amino)-2-azaspiro[3.3]heptan-2-yl)prop-2-en-1-one |
| “A108” | 1-(6-((6-amino-5-(4-phenoxyphenyl)pyrimidin-4-yl)amino)-2- |
| | azaspiro[3.3]heptan-2-yl)but-2-yn-1-one |
| “A109” | 1-((3S,4S)-4-(((6-amino-5-(1-benzyl-1H-pyrazol-4-yl)pyrimidin-4- |
| | yl)amino)methyl)-3-hydroxypiperidin-1-yl)prop-2-en-1-one |
| “A110” | 1-((3S,4S)-4-(((6-amino-5-(1-benzyl-1H-pyrazol-4-yl)pyrimidin-4- |
| | yl)amino)methyl)-3-hydroxypiperidin-1-yl)prop-2-yn-1-one |
| “A111” | 1-(6-((6-amino-5-(1-benzyl-1H-pyrazol-4-yl)pyrimidin-4- |
| | yl)amino)-2-azaspiro[3.3]heptan-2-yl)prop-2-yn-1-one |
| “A112” | 1-(6-((6-amino-5-(1-benzyl-1H-pyrazol-4-yl)pyrimidin-4-yl)oxy)-2- |
| | azaspiro[3.3]heptan-2-yl)prop-2-yn-1-one |
| “A113” | 1-(2-((6-amino-5-(4-phenoxyphenyl)pyrimidin-4-yl)amino)-6- |
| | azaspiro[3.4]octan-6-yl)prop-2-en-1-one |
| “A114” | 1-(6-((6-amino-5-(4-(pyridin-4-yloxy)phenyl)pyrimidin-4-yl)oxy)-2- |
| | azaspiro[3.3]heptan-2-yl)prop-2-en-1-one |
| “A115” | 1-(2-((6-amino-5-(4-phenoxyphenyl)pyrimidin-4-yl)amino)-6- |
| | azaspiro[3.4]octan-6-yl)prop-2-yn-1-one |
| “A116” | 1-(6-((6-amino-5-(1-(pyridin-4-ylmethyl)-1H-pyrazol-4- |
| | yl)pyrimidin-4-yl)amino)-2-azaspiro[3.3]heptan-2-yl)prop-2-en-1-one |
| “A117” | N-(1,3-trans-3-((6-amino-5-(4-phenoxyphenyl)pyrimidin-4- |
| | yl)amino)cyclobutyl)acrylamide |
| “A118” | N-((1,3-cis-3-((6-amino-5-(4-phenoxyphenyl)pyrimidin-4- |
| | yl)amino)cyclobutyl)acrylamide |
| “A119” | N4-(2-((2-chloroethyl)sulfonyl)-2-azaspiro[3.3]heptan-6-yl)-5-(4- |
| | phenoxyphenyl)pyrimidine-4,6-diamine |
| “A120” | 1-(6-((6-amino-5-(4-phenoxyphenyl)pyrimidin-4-yl)oxy)-2- |
| | azaspiro[3.3]heptan-2-yl) prop-2-en-1-one |
| “A121” | 1-(4-(((6-amino-5-(4-phenoxyphenyl)pyrimidin-4- |
| | yl)amino)methyl)-4-methoxypiperidin-1-yl)prop-2-en-1-one |
| “A122” | N-(6-((6-amino-5-(4-phenoxyphenyl)pyrimidin-4- |
| | yl)amino)spiro[3.3]heptan-2-yl)acrylamide |
| “A123” | 1-(1-((6-amino-5-(4-phenoxyphenyl)pyrimidin-4-yl)amino)-7- |
| | azaspiro[3.5]nonan-7-yl)prop-2-en-1-one |
| “A124” | 1-(6-((6-amino-5-(4-phenoxyphenyl)pyrimidin-4-yl)amino)-2- |
| | azaspiro[3.3]heptan-2-yl)prop-2-en-1-one |
| “A125” | 1-(8-((6-amino-5-(4-phenoxyphenyl)pyrimidin-4-yl)amino)-5- |
| | azaspiro[3.5]nonan-5-yl)prop-2-en-1-one |
| “A126” | (E)-1-((3S,4S)-4-(((6-amino-5-(4-phenoxyphenyl)pyrimidin-4- |
| | yl)amino)methyl)-3-hydroxypiperidin-1-yl)-4-(dimethylamino)but- |
| | 2-en-1-one |
| “A127” | (E)-1-(6-((6-amino-5-(4-phenoxyphenyl)pyrimidin-4-yl)amino)-2- |
| | azaspiro[3.3]heptan-2-yl)-4-(dimethylamino)but-2-en-1-one |
| “A128” | 3-((6-Amino-5-chloro-pyrimidin-4-ylamino)-methyl)-benzoic acid |
| | methyl ester |
| “A129” | Trans-3-(6-Amino-5-chloro-pyrimidin-4-ylamino)- |
| | cyclohexanecarboxylic acid methyl ester |
| “A130” | (1R,3S)-3-(6-Amino-5-chloro-pyrimidin-4-ylamino)- |
| | cyclohexanecarboxylic acid methyl ester |
| “A131” | 3-((6-Amino-5-(4-phenoxy-phenyl)-pyrimidin-4-ylamino)-methyl)- |
| | benzoic acid methyl ester |
| “A132” | Trans-3-(6-amino-5-(4-phenoxyphenyl)-pyrimidin-4-ylamino)- |
| | cyclohexanecarboxylic acid methyl ester |
| “A133” | (1R,3S)-3-(6-Amino-5-(4-phenoxyphenyl)-pyrimidin-4-ylamino)- |
| | cyclohexanecarboxylic acid methyl ester |
| “A134” | -((6-Amino-5-(4-phenoxy-phenyl)-pyrimidin-4-ylamino)-methyl)- |
| | benzoic acid |
| “A135” | (1S,3S)-3-(6-Amino-5-(4-phenoxyphenyl)-pyrimidin-4-ylamino)- |
| | cyclohexanecarboxylic acid |
| “A136” | (1R,3S)-3-(6-Amino-5-(4-phenoxyphenyl)-pyrimidin-4-ylamino)- |
| | cyclohexanecarboxylic acid |
| “A137” | (4-(6-Amino-5-(4-phenoxyphenyl)-pyrimidin-4-ylamino)-phenyl)- |
| | N-methoxy-N-methyl-acetamide |
| “A138” | 3-((6-Amino-5-(4-phenoxy-phenyl)-pyrimidin-4-ylamino)-methyl)- |
| | N-methoxy-N-methyl-benzamide |
| “A139” | (1S,3S)-3-(6-Amino-5-(4-phenoxyphenyl)-pyrimidin-4-ylamino)- |
| | cyclohexanecarboxylic acid methoxy-methyl-amide |
| “A140” | (1R,3S)-3-(6-Amino-5-(4-phenoxyphenyl)-pyrimidin-4-ylamino)- |
| | cyclohexanecarboxylic acid methoxy-methyl-amide |
| “A141” | 1-(3-((6-Amino-5-(4-phenoxyphenyl)-pyrimidin-4-ylamino)- |
| | methyl)-phenyl)-but-2-yn-1-one |
| “A142” | 1-(3-((6-Amino-5-(4-phenoxyphenyl)-pyrimidin-4-ylamino)- |
| | methyl)-phenyl)-but-2-en-1-one |
| “A143” | 1-((1S,3S)-3-(6-Amino-5-(4-phenoxyphenyl)-pyrimidin-4- |
| | ylamino)-cyclohexyl)-propenone |
| “A144” | 1-((1S,3S)-3-(6-Amino-5-(4-phenoxyphenyl)-pyrimidin-4- |
| | ylamino)-cyclohexyl)-but-2-en-1-one |
| “A145” | 1-((1S,3S)-3-(6-Amino-5-(4-phenoxyphenyl)-pyrimidin-4- |
| | ylamino)-cyclohexyl)-but-2-yn-1-one |
| “A146” | 1-((1S,3R)-3-(6-Amino-5-(4-phenoxyphenyl)-pyrimidin-4- |
| | ylamino)-cyclohexyl)-but-2-en-1-one |
| “A147” | 1-((1S,3R)-3-(6-amino-5-(4-phenoxyphenyl)-pyrimidin-4- |
| | ylamino)-cyclohexyl)-but-2-yn-1-one |
| “A148” | (S)-1-(3-((6-amino-5-(4-phenoxyphenyl)pyrimidin-4- |
| | yl)amino)pyrrolidin-1-yl)prop-2-en-1-one |
| “A149” | N-(3-((2-amino-3-(4-(benzyloxy)phenyl)pyridin-4- |
| | yl)oxy)phenyl)acrylamide |
| “A150” | 1-(3-((2-amino-5-(4-phenoxyphenyl)pyrimidin-4- |
| | yl)amino)piperidin-1-yl)prop-2-en-1-one |
| “A151” | (E)-N-(3-((2-amino-3-(4-phenoxyphenyl)pyridin-4-yl)oxy)phenyl)- |
| | 4-(dimethylamino)but-2-enamide |
| “A152” | (E)-N-(3-((2-amino-3-(4-(benzyloxy)phenyl)pyridin-4- |
| | yl)oxy)phenyl)-4-(dimethylamino)but-2-enamide |
| “A153” | (E)-1-(4-(((6-amino-5-(4-phenoxyphenyl)pyrimidin-4- |
| | yl)amino)methyl)piperidin-1-yl)-4-(dimethylamino)but-2-en-1-one |
| “A154” | N-cis-4-((6-amino-5-(4-(benzyloxy)phenyl)pyrimidin-4- |
| | yl)amino)cyclohexyl)acrylamide |
| “A155” | 4-(4-(((1-acryloylpyrrolidin-3-yl)methyl)amino)-6-aminopyrimidin- |
| | 5-yl)-N-phenylbenzamide |
| “A156” | 1-(3-(((6-amino-5-(4-(benzyloxy)phenyl)pyrimidin-4- |
| | yl)amino)methyl)pyrrolidin-1-yl)prop-2-en-1-one |
| “A157” | 4-(4-(((1-acryloylpiperidin-4-yl)methyl)amino)-6-aminopyrimidin- |
| | 5-yl)-N-phenylbenzamide |
| “A158” | N-(3-((2-amino-3-(4-phenoxyphenyl)pyridin-4-yl)oxy)-4- |
| | fluorophenyl)acrylamide |
| “A159” | 4-(4-((cis-4-acrylamidocyclohexyl)amino)-6-aminopyrimidin-5-yl)- |
| | N-phenylbenzamide |
| “A160” | (E)-1-(3-((6-amino-5-(4-phenoxyphenyl)pyrimidin-4- |
| | yl)amino)piperidin-1-yl)-4-(dimethylamino)but-2-en-1-one |
| “A161” | N-(3-((6-amino-5-(6-phenoxypyridin-3-yl)pyrimidin-4-yl)oxy)-4- |
| | fluorophenyl)acrylamide |
| “A162” | N-(3-((6-amino-5-(4-(pyridin-2-yloxy)phenyl)pyrimidin-4- |
| | yl)oxy)phenyl)acrylamide |
| “A163” | N-(3-((6-amino-5-(3-sulfamoylphenyl)pyrimidin-4- |
| | yl)oxy)phenyl)acrylamide |
| “A164” | N-(3-((6-amino-5-(3-(trifluoromethoxy)phenyl)pyrimidin-4- |
| | yl)oxy)phenyl)acrylamide |
| “A165” | N-(3-((6-amino-5-(6-(2-fluorophenoxy)pyridin-3-yl)pyrimidin-4- |
| | yl)oxy)phenyl)acrylamide |
| “A166” | N-(3-((6-amino-5-(6-(4-fluorophenoxy)pyridin-3-yl)pyrimidin-4- |
| | yl)oxy)phenyl)acrylamide |
| “A167” | N-(6-((5-(4-phenoxyphenyl)pyrimidin-4-yl)amino)pyridin-2- |
| | yl)acrylamide |
| “A168” | 1-(4-(((6-amino-5-(6-phenoxypyridin-3-yl)pyrimidin-4- |
| | yl)amino)methyl)piperidin-1-yl)prop-2-en-1-one |
| “A169” | 1-(4-(((6-amino-5-(4-(3- |
| | (trifluoromethyl)phenoxy)phenyl)pyrimidin-4-yl)amino)methyl)-4- |
| | hydroxypiperidin-1-yl)prop-2-en-1-one |
| “A170” | 1-((3S,4S)-4-(((6-amino-5-(4-(3- |
| | (trifluoromethyl)phenoxy)phenyl)pyrimidin-4-yl)amino)methyl)-3- |
| | hydroxypiperidin-1-yl)prop-2-en-1-one |
| “A171” | 1-(4-(((6-amino-2′-phenoxy-[5,5′-bipyrimidin]-4- |
| | yl)amino)methyl)piperidin-1-yl)prop-2-en-1-one |
| “A172” | N-(3-((6-amino-5-(1-benzyl-1H-pyrazol-4-yl)pyrimidin-4- |
| | yl)oxy)phenyl)acrylamide |
| “A173” | N-((1S,3R)-3-((6-amino-5-(4-phenoxyphenyl)pyrimidin-4- |
| | yl)amino)cyclohexyl)acrylamide |
| “A174” | N-((1R,3S)-3-((6-amino-5-(4-phenoxyphenyl)pyrimidin-4- |
| | yl)amino)cyclohexyl)acrylamide |
| “A175” | N-((1R,3R)-3-((6-amino-5-(4-phenoxyphenyl)pyrimidin-4- |
| | yl)amino)cyclohexyl)acrylamide |
| “A176” | N-((1S,3S)-3-((6-amino-5-(4-phenoxyphenyl)pyrimidin-4- |
| | yl)amino)cyclohexyl)acrylamide |
| “A177” | N-(4-((6-amino-5-(4-phenoxyphenyl)pyrimidin-4- |
| | yl)amino)bicyclo[2.1.1]hexan-1-yl)acrylamide |
| “A178” | (R)-N4-(1-((perfluorophenyl)sulfonyl)pyrrolidin-3-yl)-5-(4- |
| | phenoxyphenyl)pyrimidine-4,6-diamine |
| “A179” | (R)-N4-(1-((perfluorophenyl)sulfonyl)piperidin-3-yl)-5-(4- |
| | phenoxyphenyl)pyrimidine-4,6-diamine |
| “A180” | (R)-1-(3-((6-amino-5-(1-benzyl-1H-pyrazol-4-yl)pyrimidin-4- |
| | yl)amino)piperidin-1-yl)prop-2-en-1-one |
| “A181” | N-(cis-3-((6-amino-5-(4-phenoxyphenyl)pyrimidin-4- |
| | yl)amino)cyclopentyl)acrylamide |
| “A182” | N-(3-(((6-amino-5-(4-phenoxyphenyl)pyrimidin-4- |
| | yl)amino)methyl)cyclobutyl)acrylamide |
| “A183” | N-(3-((6-amino-5-(1-(3,5-difluorobenzyl)-1H-pyrazol-4- |
| | yl)pyrimidin-4-yl)oxy)phenyl)acrylamide |
| “A184” | N-(3-((6-amino-5-(1-(2-fluorobenzyl)-1H-pyrazol-4-yl)pyrimidin-4- |
| | yl)oxy)phenyl)acrylamide |
| “A185” | 1-(3-((6-amino-5-(4-phenoxyphenyl)pyrimidin-4- |
| | yl)amino)azetidin-1-yl)prop-2-en-1-one |
| “A186” | N-(5-((6-amino-5-(4-phenoxyphenyl)pyrimidin-4- |
| | yl)amino)pyridin-3-yl)acrylamide |
| “A187” | N-(3-((6-amino-5-(1-(4-fluorobenzyl)-1H-pyrazol-4-yl)pyrimidin-4- |
| | yl)oxy)phenyl)acrylamide |
| “A188” | N-((1R,3S,5R)-3-((6-amino-5-(4-phenoxyphenyl)pyrimidin-4- |
| | yl)amino)-5-hydroxycyclohexyl)acrylamide (racemic) |
| “A189” | N-(5-((6-amino-5-(1-benzyl-1H-pyrazol-4-yl)pyrimidin-4- |
| | yl)oxy)pyridin-3-yl)acrylamide |
| “A190” | N-(3-((6-amino-5-(1-(3-methylbenzyl)-1H-pyrazol-4-yl)pyrimidin- |
| | 4-yl)oxy)phenyl)acrylamide |
| “A191” | N-(3-((6-amino-5-(1-(3-chlorobenzyl)-1H-pyrazol-4-yl)pyrimidin- |
| | 4-yl)oxy)phenyl)acrylamide |
| “A192” | (R)-1-(2-(((6-amino-5-(1-benzyl-1H-pyrazol-4-yl)pyrimidin-4- |
| | yl)oxy)methyl)morpholino)prop-2-en-1-one |
| “A193” | (S)-1-(2-(((6-amino-5-(1-benzyl-1H-pyrazol-4-yl)pyrimidin-4- |
| | yl)oxy)methyl)morpholino)prop-2-en-1-one |
| “A194” | N-(3-((6-amino-5-(1-(2-cyanobenzyl)-1H-pyrazol-4-yl)pyrimidin- |
| | 4-yl)oxy)phenyl)acrylamide |
| “A195” | N-(3-((6-amino-5-(1-(3-(trifluoromethyl)benzyl)-1H-pyrazol-4- |
| | yl)pyrimidin-4-yl)oxy)phenyl)acrylamide |
| “A196” | (R)-1-(3-(((6-amino-5-(1-benzyl-1H-pyrazol-4-yl)pyrimidin-4- |
| | yl)oxy)methyl)pyrrolidin-1-yl)prop-2-en-1-one |
| “A197” | N-(5-((6-amino-5-(4-(4-cyanophenoxy)phenyl)pyrimidin-4- |
| | yl)oxy)pyridin-3-yl)acrylamide |
| “A198” | N-(3-((6-amino-5-(1-(3-methoxybenzyl)-1H-pyrazol-4- |
| | yl)pyrimidin-4-yl)oxy)phenyl)acrylamide |
| “A199” | 4-(4-(4-((((3S,4S)-1-acryloyl-3-hydroxypiperidin-4- |
| | yl)methyl)amino)-6-aminopyrimidin-5-yl)phenoxy)benzonitrile |
| “A200” | (R)-4-(4-(4-((4-acryloylmorpholin-2-yl)methoxy)-6- |
| | aminopyrimidin-5-yl)phenoxy)benzonitrile |
| “A201” | (R)-4-(4-(4-((1-acryloylpyrrolidin-3-yl)methoxy)-6- |
| | aminopyrimidin-5-yl)phenoxy)benzonitrile |
| “A202” | 4-(4-(4-((2-acryloyl-2-azaspiro[3.3]heptan-6-yl)oxy)-6- |
| | aminopyrimidin-5-yl)phenoxy)benzonitrile |
| “A203” | N-(3-((6-amino-5-(1-(3-cyanobenzyl)-1H-pyrazol-4-yl)pyrimidin- |
| | 4-yl)oxy)phenyl)acrylamide |
| “A204” | 1-((3S,5S)-3-((6-amino-5-(4-phenoxyphenyl)pyrimidin-4- |
| | yl)amino)-5-fluoropiperidin-1-yl)prop-2-en-1-one |
| “A205” | 1-((3R,5R)-3-((6-amino-5-(4-phenoxyphenyl)pyrimidin-4- |
| | yl)amino)-5-fluoropiperidin-1-yl)prop-2-en-1-one |
| “A206” | methyl 3-((4-(4-(3-acrylamidophenoxy)-6-aminopyrimidin-5-yl)- |
| | 1H-pyrazol-1-yl)methyl)benzoate |
| “A207” | 4-(4-(4-((2-acryloyl-2-azaspiro[3.3]heptan-6-yl)amino)-6- |
| | aminopyrimidin-5-yl)phenoxy)benzonitrile |
| “A208” | 4-(4-(4-(((8-acryloyl-8-azabicyclo[3.2.1]octan-3- |
| | yl)methyl)amino)-6-aminopyrimidin-5-yl)phenoxy)benzonitrile |
| “A209” | 1-(3-(((6-amino-5-(1-benzyl-1H-pyrazol-4-yl)pyrimidin-4- |
| | yl)amino)methyl)-8-azabicyclo[3.2.1]octan-8-yl)prop-2-en-1-one |
| “A210” | 1-((3R,4R)-3-((6-amino-5-(4-phenoxyphenyl)pyrimidin-4- |
| | yl)amino)-4-hydroxypiperidin-1-yl)prop-2-en-1-one (racemic) |
| “A211” | N-(3-((6-amino-5-(1-(3-(methylsulfonyl)benzyl)-1H-pyrazol-4- |
| | yl)pyrimidin-4-yl)oxy)phenyl)acrylamide |
| “A212” | N-(3-((6-amino-5-(1-(3-(dimethylamino)benzyl)-1H-pyrazol-4- |
| | yl)pyrimidin-4-yl)oxy)phenyl)acrylamide |
| “A213” | N-(3-((6-amino-5-(4-(3-cyanophenoxy)phenyl)pyrimidin-4- |
| | yl)oxy)phenyl)acrylamide |
| “A214” | 3-(4-(4-(((1-acryloylpiperidin-4-yl)methyl)amino)-6- |
| | aminopyrimidin-5-yl)phenoxy)benzonitrile |
| “A215” | 1-((3S,4S)-4-(((6-amino-5-(4-phenoxyphenyl)pyrimidin-4- |
| | yl)amino)methyl)-3-hydroxypiperidin-1-yl)but-2-yn-1-one |
| “A216” | 1-acryloyl-4-(((6-amino-5-(4-phenoxyphenyl)pyrimidin-4- |
| | yl)amino)methyl)piperidine-4-carboxylic acid |
| “A217” | (E)-4-(((6-amino-5-(4-phenoxyphenyl)pyrimidin-4- |
| | yl)amino)methyl)-1-(4-(dimethylamino)but-2-enoyl)piperidine-4- |
| | carboxylic acid |
| “A218” | (E)-1-(4-(((6-amino-5-(4-phenoxyphenyl)pyrimidin-4- |
| | yl)amino)methyl)piperidin-1-yl)-4-(3-fluoroazetidin-1-yl)but-2-en- |
| | 1-one |
| “A219” | (E)-1-(4-(((6-amino-5-(4-phenoxyphenyl)pyrimidin-4- |
| | yl)amino)methyl)piperidin-1-yl)-4-(3,3-difluoroazetidin-1-yl)but-2- |
| | en-1-one |
| “A220” | (E)-1-(4-(((6-amino-5-(4-phenoxyphenyl)pyrimidin-4- |
| | yl)amino)methyl)piperidin-1-yl)-4-(pyrrolidin-1-yl)but-2-en-1-one |
| “A221” | 1-(6-((6-amino-5-(4-(pyridin-3-yloxy)phenyl)pyrimidin-4-yl)oxy)-2- |
| | azaspiro[3.3]heptan-2-yl)prop-2-en-1-one |
| “A222” | (E)-1-(6-((6-amino-5-(4-phenoxyphenyl)pyrimidin-4-yl)oxy)-2- |
| | azaspiro[3.3]heptan-2-yl)-4-(3-fluoroazetidin-1-yl)but-2-en-1-one |
| “A223” | (E)-1-(6-((6-amino-5-(4-phenoxyphenyl)pyrimidin-4-yl)oxy)-2- |
| | azaspiro[3.3]heptan-2-yl)-4-(3-fluoroazetidin-1-yl)but-2-en-1-one |
| “A224” | (E)-N-(1,3-cis-3-((6-amino-5-(4-phenoxyphenyl)pyrimidin-4- |
| | yl)amino)cyclobutyl)-4-(dimethylamino)but-2-enamide |
| “A225” | 1-(4-(((6-amino-5-(4-phenoxyphenyl)pyrimidin-4- |
| | yl)amino)methyl)-4-fluoropiperidin-1-yl)prop-2-en-1-one; |
| | compound (2) |
| “A226” | (E)-1-(2-((6-amino-5-(4-phenoxyphenyl)pyrimidin-4-yl)amino)-6- |
| | azaspiro[3.4]octan-6-yl)-4-(dimethylamino)but-2-en-1-one |
| “A227” | (E)-1-(4-(((6-amino-5-(4-phenoxyphenyl)pyrimidin-4- |
| | yl)amino)methyl)-4-fluoropiperidin-1-yl)-4-(dimethylamino)but-2- |
| | en-1-one |
| “A228” | (E)-N-(1,3-trans-3-((6-amino-5-(4-phenoxyphenyl)pyrimidin-4- |
| | yl)amino)cyclobutyl)-4-(dimethylamino)but-2-enamide |
| “A229” | N-(1,3-cis-3-((6-amino-5-(1-benzyl-1H-pyrazol-4-yl)pyrimidin-4- |
| | yl)amino)cyclobutyl)acrylamide |
| “A230” | (E)-N-(1,3-cis-3-((6-amino-5-(1-benzyl-1H-pyrazol-4-yl)pyrimidin- |
| | 4-yl)amino)cyclobutyl)-4-(dimethylamino)but-2-enamide |
| “A231” | (E)-1-(4-(((6-amino-5-(4-phenoxyphenyl)pyrimidin-4- |
| | yl)amino)methyl)piperidin-1-yl)-3-phenylprop-2-en-1-one |
| “A232” | 1-((3S,4S)-4-(((6-amino-5-(4-phenoxyphenyl)pyrimidin-4- |
| | yl)amino)methyl)-3-hydroxypiperidin-1-yl)-3- |
| | (dimethylamino)propan-1-one |
| “A233” | 1-((3S,4S)-4-(((6-amino-5-(4-phenoxyphenyl)pyrimidin-4- |
| | yl)amino)methyl)-3-hydroxypiperidin-1-yl)-3-(piperidin-1- |
| | yl)propan-1-one |
| “A234” | 1-((3S,4S)-4-(((6-amino-5-(4-phenoxyphenyl)pyrimidin-4- |
| | yl)amino)methyl)-3-hydroxypiperidin-1-yl)-3-morpholinopropan- |
| | 1-one |
| “A235” | 1-(4-(((6-amino-5-(4-phenoxyphenyl)pyrimidin-4- |
| | yl)amino)methyl)-4-fluoropiperidin-1-yl)-3-(piperidin-1-yl)propan- |
| | 1-one |
| “A236” | (E)-N-(1,3-cis-3-((6-amino-5-(4-(3- |
| | (trifluoromethyl)phenoxy)phenyl)pyrimidin-4- |
| | yl)amino)cyclobutyl)-4-(dimethylamino)but-2-enamide |
| “A237” | N-(1,3-trans-3-((6-amino-5-(4-(3- |
| | (trifluoromethyl)phenoxy)phenyl)pyrimidin-4- |
| | yl)amino)cyclobutyl)acrylamide |
| “A238” | N-(1,3-cis-3-((6-amino-5-(4-(3- |
| | (trifluoromethyl)phenoxy)phenyl)pyrimidin-4- |
| | yl)amino)cyclobutyl)acrylamide |
| “A239” | 1-acryloyl-4-(((6-amino-5-(4-(3- |
| | (trifluoromethyl)phenoxy)phenyl)pyrimidin-4- |
| | yl)amino)methyl)piperidine-4-carboxylic acid |
| “A240” | N-(3-((6-amino-5-(4-phenoxyphenyl)pyrimidin-4-yl)oxy)-2- |
| | fluorophenyl)acrylamide |
| “A241” | N-(3-(4-amino-6-((4-phenoxyphenyl)amino)pyrimidin-5- |
| | yl)phenyl)acrylamide |
| “A242” | N-(3-((6-amino-5-(4-phenoxyphenyl)pyrimidin-4- |
| | yl)oxy)phenyl)acrylamide |
| “A243” | N-(3-(2-amino-4-(4-phenoxyphenoxy)pyridin-3- |
| | yl)phenyl)acrylamide |
| “A244” | N-(3-((2-amino-3-(4-phenoxyphenyl)pyridin-4- |
| | yl)oxy)phenyl)acrylamide |
| “A245” | N-(3-(4-amino-6-(4-phenoxyphenoxy)pyrimidin-5- |
| | yl)phenyl)acrylamide |
| “A246” | N-(3-((6-amino-5-(4-phenoxyphenyl)pyrimidin-4-yl)oxy)-4- |
| | fluorophenyl)acrylamide |
| “A247” | (R)-1-(3-((6-amino-5-(4-phenoxyphenyl)pyrimidin-4- |
| | yl)amino)piperidin-1-yl)prop-2-en-1-one |
| “A248” | (E)-N-(3-(4-amino-6-(4-phenoxyphenoxy)pyrimidin-5-yl)phenyl)- |
| | 4-(dimethylamino)but-2-enamide |
| “A249” | N-(3-((6-amino-5-(4-(benzyloxy)phenyl)pyrimidin-4- |
| | yl)oxy)phenyl)acrylamide |
| “A250” | 1-(4-(((6-amino-5-(4-phenoxyphenyl)pyrimidin-4- |
| | yl)amino)methyl)piperidin-1-yl)prop-2-en-1-one; compound (1) |
| “A251” | N-(5-((6-amino-5-(4-phenoxyphenyl)pyrimidin-4-yl)oxy)-2,4- |
| | difluorophenyl)acrylamide |
| “A252” | (E)-N-(3-((6-amino-5-(4-phenoxyphenyl)pyrimidin-4- |
| | yl)oxy)phenyl)-4-(dimethylamino)but-2-enamide |
| “A253” | 1-(3-((6-amino-5-(4-phenoxyphenyl)pyrimidin-4- |
| | yl)amino)piperidin-1-yl)prop-2-en-1-one |
| “A254” | N-(3-((6-amino-5-(4-((2-methoxybenzyl)oxy)phenyl)pyrimidin-4- |
| | yl)oxy)phenyl)acrylamide |
| “A255” | N-(3-((5-(4-phenoxyphenyl)pyrimidin-4-yl)oxy)phenyl)acrylamide |
| “A256” | N-(3-((6-amino-5-(4-(benzyloxy)-3-methoxyphenyl)pyrimidin-4- |
| | yl)oxy)phenyl)acrylamide |
| “A257” | N-(3-((6-amino-5-(4-(benzyloxy)-2,3-difluorophenyl)pyrimidin-4- |
| | yl)oxy)phenyl)acrylamide |
| “A258” | 4-(4-(3-acrylamidophenoxy)-6-aminopyrimidin-5-yl)-N- |
| | phenylbenzamide |
| “A259” | N-(3-((6-amino-5-(6-(benzyloxy)pyridin-3-yl)pyrimidin-4- |
| | yl)oxy)phenyl)acrylamide |
| “A260” | N-(3-((6-amino-5-(4-((3-fluorobenzyl)oxy)phenyl)pyrimidin-4- |
| | yl)oxy)phenyl)acrylamide |
| “A261” | N-(3-((6-amino-2′-(benzyloxy)-[5,5′-bipyrimidin]-4- |
| | yl)oxy)phenyl)acrylamide |
| “A262” | 1-(3-(((6-amino-5-(4-phenoxyphenyl)pyrimidin-4- |
| | yl)amino)methyl)pyrrolidin-1-yl)prop-2-en-1-one |
| “A263” | 1-(4-(4-amino-6-(4-phenoxyphenoxy)pyrimidin-5-yl)-5,6- |
| | dihydropyridin-1(2H)-yl)prop-2-en-1-one |
| “A264” | N-(3-((6-amino-5-(4-((4-methoxybenzyl)oxy)phenyl)pyrimidin-4- |
| | yl)oxy)phenyl)acrylamide |
| “A265” | (E)-N-(3-((6-amino-5-(4-phenoxyphenyl)pyrimidin-4- |
| | yl)oxy)phenyl)-4-morpholinobut-2-enamide |
| “A266” | N-((1s,4s)-4-((6-amino-5-(4-phenoxyphenyl)pyrimidin-4- |
| | yl)amino)cyclohexyl)acrylamide |
| “A267” | N-(3-(4-((4-phenoxyphenyl)amino)pyridin-3-yl)phenyl)acrylamide |
| “A268” | N-(3-((6-amino-5-(6-phenoxypyridin-3-yl)pyrimidin-4- |
| | yl)oxy)phenyl)acrylamide |
| “A269” | 1-(3-((6-amino-5-(4-(benzyloxy)phenyl)pyrimidin-4- |
| | yl)amino)piperidin-1-yl)prop-2-en-1-one |
| “A270” | N-(3-((3-(4-phenoxyphenyl)pyridin-4-yl)oxy)phenyl)acrylamide |
| “A271” | N-(3-((2-amino-5-(4-phenoxyphenyl)pyrimidin-4- |
| | yl)oxy)phenyl)acrylamide |
| “A272” | 3-(3-acrylamidophenyl)-4-(4-phenoxyphenoxy)picolinamide |
| “A273” | 1-(3-(4-amino-6-((4-phenoxyphenyl)amino)pyrimidin-5-yl)-5,6- |
| | dihydropyridin-1(2H)-yl)prop-2-en-1-one |
| “A274” | (E)-N-(3-(4-amino-6-(4-phenoxyphenoxy)pyrimidin-5-yl)phenyl)- |
| | 4-morpholinobut-2-enamide |
| “A275” | (S)-1-(3-((6-amino-5-(4-phenoxyphenyl)pyrimidin-4- |
| | yl)amino)piperidin-1-yl)prop-2-en-1-one |
| “A276” | N-((1r,4r)-4-((6-amino-5-(4-phenoxyphenyl)pyrimidin-4- |
| | yl)amino)cyclohexyl)acrylamide |
| “A277” | N-(3-((6-amino-5-(4-fluoro-3-methoxyphenyl)pyrimidin-4- |
| | yl)oxy)phenyl)acrylamide |
| “A278” | N-(3-((6-amino-5-(4-(2-hydroxypropan-2-yl)phenyl)pyrimidin-4- |
| | yl)oxy)phenyl)acrylamide |
| “A279” | 1-(3-(4-amino-6-(4-phenoxyphenoxy)pyrimidin-5-yl)-5,6- |
| | dihydropyridin-1(2H)-yl)prop-2-en-1-one |
| “A280” | N-(4-((6-amino-5-(4-phenoxyphenyl)pyrimidin-4- |
| | yl)oxy)phenyl)acrylamide |
| “A281” | N-(4-(4-(4-phenoxyphenoxy)pyridin-3-yl)phenyl)acrylamide |
| “A282” | (E)-4-(dimethylamino)-N-(3-(4-(4-phenoxyphenoxy)pyridin-3- |
| | yl)phenyl)but-2-enamide |
| “A283” | N-(3-(4-((4-phenoxyphenyl)amino)pyrimidin-5- |
| | yl)phenyl)acrylamide |
| “A284” | 1-(3-((5-(4-phenoxyphenyl)pyrimidin-4-yl)amino)piperidin-1- |
| | yl)prop-2-en-1-one |
| “A285” | N-(3-((6-amino-5-(4-(pyrrolidine-1-carbonyl)phenyl)pyrimidin-4- |
| | yl)oxy)phenyl)acrylamide |
| “A286” | 1-(3-(((6-amino-5-(4-phenoxyphenyl)pyrimidin-4- |
| | yl)amino)methyl)piperidin-1-yl)prop-2-en-1-one |
| “A287” | N-(4-(4-(4-phenoxyphenoxy)pyridin-3-yl)benzyl)acrylamide |
| “A288” | 1-(4′-(4-phenoxyphenoxy)-5,6-dihydro-[3,3′-bipyridin]-1(2H)- |
| | yl)prop-2-en-1-one |
| “A289” | N-(3-((6-amino-5-(4-isopropoxyphenyl)pyrimidin-4- |
| | yl)oxy)phenyl)acrylamide |
| “A290” | (E)-N-(4-((6-amino-5-(4-phenoxyphenyl)pyrimidin-4- |
| | yl)oxy)phenyl)-4-(dimethylamino)but-2-enamide |
| “A291” | N-(3-((6-amino-5-(5-methoxypyridin-3-yl)pyrimidin-4- |
| | yl)oxy)phenyl)acrylamide |
| “A292” | 1-(4-(((6-amino-5-(4-(benzyloxy)phenyl)pyrimidin-4- |
| | yl)amino)methyl)piperidin-1-yl)prop-2-en-1-one |
| “A293” | (E)-4-morpholino-N-(3-(4-(4-phenoxyphenoxy)pyridin-3- |
| | yl)phenyl)but-2-enamide |
| “A294” | N-(3-((6-amino-5-(4-(benzyloxy)-2,6-difluorophenyl)pyrimidin-4- |
| | yl)oxy)phenyl)acrylamide |
| “A295” | (E)-N-(3-((6-amino-5-(4-phenoxyphenyl)pyrimidin-4- |
| | yl)oxy)phenyl)-4-(4-(5-((4S)-2-oxohexahydro-1H-thieno[3,4- |
| | d]imidazol-4-yl)pentanoyl)piperazin-1-yl)but-2-enamide |
| “A296” | N-(3-(4-(4-phenoxyphenoxy)pyridin-3-yl)phenyl)but-2-ynamide |
| “A297” | N-(4-((3-(4-phenoxyphenyl)pyridin-4-yl)oxy)phenyl)acrylamide |
| “A298” | N-(1-(6-amino-5-(4-phenoxyphenyl)pyrimidin-4-yl)piperidin-3- |
| | yl)acrylamide |
| “A299” | 1-(4-((6-amino-5-(4-phenoxyphenyl)pyrimidin-4- |
| | yl)amino)piperidin-1-yl)prop-2-en-1-one |
| “A300” | 3-(3-aminophenyl)-4-(4-phenoxyphenoxy)pyridin-2-amine |
| “A301” | (E)-N-(3-(4-amino-6-(4-phenoxyphenoxy)pyrimidin-5-yl)phenyl)- |
| | 4-(3,3-difluoropiperidin-1-yl)but-2-enamide |
| “A302” | N-(3-(4-(4-phenoxyphenoxy)pyridin-3-yl)benzyl)acrylamide |
| “A303” | 6-(4-aminophenoxy)-5-(4-phenoxyphenyl)pyrimidin-4-amine |
| “A304” | N-(3-(4-(4-phenoxyphenoxy)pyridin-3-yl)benzyl)but-2-ynamide |
| “A305” | 6-(3-aminophenoxy)-5-(4-phenoxyphenyl)pyrimidin-4-amine |
| “A306” | N-(3-(2-amino-4-(4-phenoxyphenoxy)pyrimidin-5- |
| | yl)phenyl)acrylamide |
| “A307” | (E)-N-(3-(4-(4-phenoxyphenoxy)pyridin-3-yl)phenyl)but-2- |
| | enamide |
| “A308” | N-(4-((6-amino-5-(4-phenoxyphenyl)pyrimidin-4- |
| | yl)oxy)phenyl)propionamide |
| “A309” | N-((1-(6-amino-5-(4-phenoxyphenyl)pyrimidin-4-yl)piperidin-3- |
| | yl)methyl)acrylamide |
| “A310” | N-(3-(2-amino-4-(4-phenoxyphenoxy)pyridin-3- |
| | yl)phenyl)propionamide |
| “A311” | (R)-N-(3-(4-amino-6-((1-phenylethyl)amino)pyrimidin-5- |
| | yl)phenyl)acrylamide |
| “A312” | 3-(4-(4-phenoxyphenoxy)pyridin-3-yl)aniline |
| “A313” | 4-(3-aminophenoxy)-3-(4-phenoxyphenyl)pyridin-2-amine |
| “A314” | 4-(4-(4-phenoxyphenoxy)pyridin-3-yl)aniline |
| “A315” | (4-(4-(4-phenoxyphenoxy)pyridin-3-yl)phenyl)methanamine |
| “A316” | (3-(4-(4-phenoxyphenoxy)pyridin-3-yl)phenyl)methanamine |
| “A317” | 5-(3-aminophenyl)-6-(4-phenoxyphenoxy)pyrimidin-4-amine |
| “A318” | N-(3-((3-(4-phenoxyphenyl)pyridin-4-yl)oxy)phenyl)propionamide |
| “A319” | N-(3-(4-(4-phenoxyphenoxy)pyridin-3-yl)phenyl)propionamide |
| “A320” | N-(4-(4-(4-phenoxyphenoxy)pyridin-3-yl)phenyl)propionamide |
| “A321” | N-(4-((3-(4-phenoxyphenyl)pyridin-4-yl)oxy)phenyl)propionamide |
| “A322” | N-(3-(4-(4-phenoxyphenoxy)pyridin-3-yl)phenyl)methacrylamide |
| “A323” | N-(3-(4-(4-phenoxyphenoxy)pyridin-3-yl)benzyl)propionamide |
| “A324” | N-(4-(4-(4-phenoxyphenoxy)pyridin-3-yl)benzyl)propionamide |
| “A325” | N-(3-(4-amino-6-(4-phenoxyphenoxy)pyrimidin-5- |
| | yl)phenyl)propionamide |
| “A326” | N-(3-((6-amino-5-(4-phenoxyphenyl)pyrimidin-4- |
| | yl)oxy)phenyl)propionamide |
| “A327” | (E)-N-(3-(4-(4-phenoxyphenoxy)pyridin-3-yl)benzyl) |
| | but-2-enamide |
| “A328” | 3-(4-phenoxyphenyl)-4-(3-propionamidophenoxy)picolinamide |
| “A329” | N-(3-((6-amino-5-(4-phenoxyphenyl)pyrimidin-4-yl)oxy)phenyl)- |
| | 1-cyanocyclopropanecarboxamide |
| “A330” | N-(3-(4-amino-6-(4-phenoxyphenoxy)pyrimidin-5-yl)phenyl)-1- |
| | cyanocyclopropanecarboxamide |
| “A331” | (E)-3-(7-((6-amino-5-(4-phenoxyphenyl)pyrimidin-4- |
| | yl)oxy)naphthalen-2-yl)-N,N-dimethylacrylamide |
| “A332” | 1-(4-(1-((6-amino-5-(4-phenoxyphenyl)pyrimidin-4- |
| | yl)amino)ethyl)piperidin-1-yl)prop-2-en-1-one |
| “A333” | 1-(4-(((6-amino-5-(4-phenoxyphenyl)pyrimidin-4- |
| | yl)amino)methyl)piperidin-1-yl)propan-1-one |
| “A334” | 1-(4-(((5-(4-phenoxyphenyl)pyrimidin-4- |
| | yl)amino)methyl)piperidin-1-yl)prop-2-en-1-one |
| “A335” | 1-(4-(((6-amino-5-(4-(pyridin-2-yloxy)phenyl)pyrimidin-4- |
| | yl)amino)methyl)piperidin-1-yl)prop-2-en-1-one |
| “A336” | 1-(4-(((6-amino-5-(4-phenoxyphenyl)pyrimidin-4- |
| | yl)amino)methyl)piperidin-1-yl)but-2-yn-1-one |
| “A337” | N4-((1-(6-chloropyridin-2-yl)piperidin-4-yl)methyl)-5-(4- |
| | phenoxyphenyl)pyrimidine-4,6-diamine |
| “A338” | 1-(4-(((6-amino-5-(4-phenoxyphenyl)pyrimidin-4- |
| | yl)oxy)methyl)piperidin-1-yl)prop-2-en-1-one |
| “A339” | N-(3-((6-amino-5-(4-(benzyloxy)-2,5-difluorophenyl)pyrimidin-4- |
| | yl)oxy)phenyl)acrylamide |
| “A340” | N-(3-((2-amino-3-(4-phenoxyphenyl)pyridin-4-yl)oxy)phenyl)but- |
| | 2-ynamide |
| “A341” | (R)-1-(3-((6-amino-5-(4-phenoxyphenyl)pyrimidin-4- |
| | yl)amino)pyrrolidin-1-yl)but-2-yn-1-one |
| “A342” | N-{3-[6-Amino-5-(4-phenoxy-phenyl)-pyrimidin-4-yloxy]-phenyl}- |
| | 2-chloroacetamide |
| “A343” | N-(3-{6-Amino-5-[4-(2-fluoro-benzyloxy)-phenyl]-pyrimidin-4- |
| | yloxy}-phenyl)-acrylamide |
| “A344” | N-(3-{6-Amino-5-[4-(4-fluoro-benzyloxy)-phenyl]-pyrimidin-4- |
| | yloxy}-phenyl)-acrylamide |
| “A345” | N-(3-{6-Amino-5-[4-(3-fluoro-benzyloxy)-phenyl]-pyrimidin-4- |
| | yloxy}-phenyl)-2-chloro-acetamide |
| “A346” | N-{3-[6-Amino-5-(4-benzyloxy-phenyl)-pyrimidin-4-yloxy]- |
| | phenyl}-propionamide |
| “A347” | N-{3-[6-Amino-5-(4-benzyloxy-phenyl)-pyrimidin-4-yloxy]- |
| | phenyl}-2-chloro-acetamide |
| “A348” | N-{3-[6-Amino-5-(4-benzyloxy-3-fluoro-phenyl)-pyrimidin-4- |
| | yloxy]-phenyl}-acrylamide |
| “A349” | N-{3-[6-Amino-5-(4-benzyloxy-2-fluoro-phenyl)-pyrimidin-4- |
| | yloxy]-phenyl}-acrylamide |
| “A350” | N-{3-(6-Amino-5-(4-benzyloxy-2-fluoro-phenyl)-pyrimidin-4- |
| | yloxy}-phenyl)-2-chloro-acetamide |
| “A351” | N-{3-[6-Amino-5-(4-benzyloxy-3-fluoro-phenyl)-pyrimidin-4- |
| | yloxy]-phenyl}-2-chloro-acetamide |
| “A352” | N-{4-[4-(3-Acryloylamino-phenoxy)-6-amino-pyrimidin-5-yl]- |
| | phenyl}-benzamide |

In certain embodiments, the invention provides a method for the treatment and/or prophylaxis of cancer, comprising administering to a subject a compound selected from: N-[(1-acryloylpiperidin-4-yl)methyl]-5-(4-phenoxyphenyl)pyrimidine-4,6-diamine (A250); and 1-(4-(((6-amino-5-(4-phenoxyphenyl)pyrimidin-4-yl)amino)methyl)-4-fluoropiperidin-1-yl) prop-2-en-1-one (A225).

In certain embodiments, the invention provides a method as described above, wherein the compound is N-[(1-acryloylpiperidin-4-yl)methyl]-5-(4-phenoxyphenyl)pyrimidine-4,6-diamine (A250).

In certain embodiments, the invention provides a method as described above, wherein the compound is 1-(4-(((6-amino-5-(4-phenoxyphenyl)pyrimidin-4-yl)amino)methyl)-4-fluoropiperidin-1-yl)prop-2-en-1-one (A225).

In certain embodiments, the method of the invention is used to treat or prevent a condition selected from a proliferative or hyperproliferative disease, e.g., cancer.

In certain embodiments the invention provides a method of treating, preventing, or lessening the severity of cancer of a patient, by administering a compound or composition of the present invention.

In certain embodiments, the invention provides for the treatment of diseases, disorders, and conditions characterized by excessive or abnormal cell proliferation. Such diseases include a proliferative or hyperproliferative disease. Examples of proliferative and hyperproliferative diseases include cancer and myeloproliferative disorders.

In certain embodiments, the term “cancer” includes, but is not limited to the following cancers. Oral: head and neck, including buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma and teratoma; Lung: Non-small cell lung carcinoma including adenocarcinoma (acinar, bronchioloalveolar carcinoma [nonmucinous, mucinous, mixed], papillary, solid adenocarcinoma, clear cell, mucinous [colloid] adenocarcinoma, mucinous cystadenocarcinoma, signet ring, well-differentiated fetal), bronchioalveolar, squamous cell carcinoma (basaloid, clear cell, papillary, small cell), large cell (undifferentiated) carcinoma (giant cell, basaloid, clear cell, large cell [with rhabdoid phenotype], large cell neuroendocrine carcinoma [LCNEC], combined LCNEC); small cell lung cancer including small cell (oat cell) carcinoma, combined small cell; adenoid cystic carcinoma; hamartoma; lymphoma; neuroendocrine/carcinoid; sarcoma. Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal; rectum, Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Female/Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma, undifferentiated thyroid cancer, medullary thyroid carcinoma, multiple endocrine neoplasia type 2A, multiple endocrine neoplasia type 2B, familial medullary thyroid cancer, pheochromocytoma, paraganglioma; and Adrenal glands: neuroblastoma.

In certain embodiments, the cancer is selected from Non-Hodgkin's Lymphoma mantle cell lymphoma, and Non-Hodgkins's Lymphoma diffuse large b-cell lymphoma, including abc subtype. In certain embodiments, the cancer is Non-Hodgkin's Lymphoma mantle cell lymphoma. In certain embodiments, the cancer is Non-Hodgkins's Lymphoma diffuse large b-cell lymphoma. In certain embodiments, the cancer is Non-Hodgkins's Lymphoma diffuse large b-cell lymphoma, including abc subtype.

In certain embodiments, the cancer is selected from Non-Hodgkin's Lymphoma mantle cell lymphoma, and Non-Hodgkins's Lymphoma diffuse large b-cell lymphoma, including abc subtype, and the compound is A250 or A225. In certain embodiments, the compound is A250. In certain embodiments, the compound is A225. In certain embodiments, the cancer is Non-Hodgkin's Lymphoma mantle cell lymphoma and the compound is A250 or A225. In certain embodiments, the compound is A250. In certain embodiments, the compound is A225. In certain embodiments, the cancer is Non-Hodgkins's Lymphoma diffuse large b-cell lymphoma and the compound is A250 or A225. In certain embodiments, the compound is A250. In certain embodiments, the compound is A225. In certain embodiments, the cancer is Non-Hodgkins's Lymphoma diffuse large b-cell lymphoma, including abc subtype, and the compound is A250 or A225. In certain embodiments, the compound is A250. In certain embodiments, the compound is A225.

In general, all residues which occur more than once may be identical or different, i.e. are independent of one another. Above and below, the residues and parameters have the meanings indicated for Formula (I), Formula (II), Formula (III), Formula (IV) and Formula (V) unless expressly indicated otherwise. Accordingly, the invention relates, in particular, to the compounds of Formula (I), Formula (II), Formula (III), Formula (IV) and Formula (V) in which at least one of the said residues has one of the preferred meanings indicated below.

The term “substituted” preferably relates to the substitution by the above-mentioned substituents, where a plurality of different degrees of substitution are possible, unless indicated otherwise.

All physiologically acceptable salts, derivatives, solvates, solvates of salts, and stereo-isomers of these compounds, including mixtures thereof in all ratios, are also in accordance with the invention.

The compounds of the Formula (I), (II), (III), (IV) and (V) may have one or more centres of chirality. They may accordingly occur in various enantiomeric forms and be in racemic or optically active form. The invention therefore also relates to the optically active forms (stereoisomers), the enantiomers, the racemates, the diastereomers and hydrates and solvates of these compounds.

Since the pharmaceutical activity of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitably N-protected amino acids (for example N-benzoylproline or N-benzenesulfonylproline), or the various optically active camphorsulfonic acids. Also advantageous is chromatographic enantio-mer resolution with the aid of an optically active resolving agent (for example dinitrobenzoylphenylglycine, cellulose triacetate or other derivatives of carbohydrates or chirally derivatised methacrylate polymers immobilised on silica gel). Suitable eluents for this purpose are aqueous or alcoholic solvent mixtures, such as, for example, hexane/isopropanol/acetonitrile, for example in the ratio 82:15:3. An elegant method for the resolution of racemates containing ester groups (for example acetyl esters) is the use of enzymes, in particular esterases.

It is also contemplated that compounds of Formula (I), Formula (II), Formula (III), Formula (IV) and Formula (V) include isotope-labeled forms thereof. An isotope-labeled form of a compound of Formula (I), Formula (II), Formula (III), Formula (IV) and Formula (V) is identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available and which can be incorporated into a compound of the Formula I by well-known methods include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively. It is also contemplated that a compound of the Formula I, a prodrug, thereof or a pharmaceutically acceptable salt of either which contains one or more of the above-mentioned isotopes and/or other iso-topes of other atoms are embodiments of the present invention. An isotope-labeled compound of the Formula I can be used in a number of beneficial ways. For example, an isotope-labeled compound of the Formula I into which, for example, a radioisotope, such as ³H or ¹⁴C, has been incorporated, is suitable for medicament and/or substrate tissue distribution assays. These radioisotopes, i.e. tritium (³H) and carbon-14 (¹⁴C), are particularly preferred owing to their ease of preparation and excellent detectability. Incorporation of heavier isotopes, for example deuterium (²H), into a compound of the Formula I may have therapeutic advantages owing to the higher metabolic stability of this isotope-labeled compound. Higher metabolic stability translates directly into an increased in vivo half-life or lower dosages, which under some circumstances would represent a preferred embodiment of the present invention. An isotope-labeled compound of the Formula I can adapted to the procedures disclosed in the synthesis schemes and the related description, in the example part and in the preparation part in the present text, replacing a non-isotope-labeled reactant by a readily available isotope-labeled reactant.

In other embodiments it is contemplated that deuterium (²H) may be incorporated into a compound of Formula (I), Formula (II), Formula (III), Formula (IV) and Formula (V). Such deuterated compounds can modify the oxidative metabolism of said deuterated compound by means the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. If this rate difference is observed in any compounds of Formula (I), Formula (II), Formula (III), Formula (IV) and Formula (V) susceptible to oxidation, the profile of this compound, in vivo, can be drastically modified and result in improved pharmacokinetic properties.

When discovering and developing therapeutic agents, the person skilled in the art attempts to optimize pharmacokinetic parameters while retaining desirable in vitro properties. It is reasonable to assume that many compounds with poor pharmacokinetic profiles are susceptible to oxidative metabolism. In vitro liver microsomal assays known in the are may provide valuable information on the course of oxidative metabolism of this type, which in turn permits the rational design of deuterated compounds of Formula (I), Formula (II), Formula (III), Formula (IV) and Formula (V) with improved stability through resistance to said oxidative metabolism. Significant improvements in the pharmacokinetic profiles of compounds of the Formula I may thereby be obtained, and can be expressed quantitatively in terms of increases in the in vivo half-life (t/2), concentration at maximum therapeutic effect (Cₘₐₓ), area under the dose response curve (AUC), and F; and in terms of reduced clearance, dose and materials costs.

While it is not intended that the present invention be limited to any deuterated motif, the following is an example. A compound of Formula (I), Formula (II), Formula (III), Formula (IV) and Formula (V) which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favorable and accurate determination of the extent of the extent to which the improve-ment in resistance to oxidative metabolism has improved. In this way, it can be determined that the half-life of the parent compound may be extended by up to 100% as the result of deuterium-hydrogen exchange of this type.

Deuterium-hydrogen exchange in a compound of Formula (I), Formula (II), Formula (III), Formula (IV) and Formula (V) can also be used to achieve a favorable modification of the metabolite spectrum of the starting compound in order to diminish or eliminate undesired toxic metabolites. For example, if a toxic metabolite arises through oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1993.

The compounds of the present invention can be in the form of a prodrug compound. “Prodrug compound” means a derivative that is converted into a biologically active compound according to the present invention under physiological conditions in the living body, e.g., by oxidation, reduction, hydrolysis or the like, each of which is carried out enzymatically, or without enzyme involvement. Examples of prodrugs are compounds, wherein the amino group in a compound of the present invention is acylated, alkylated or phosphorylated, e.g., eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated, or wherein a sulfhydryl group forms a disulfide bridge with a carrier molecule, e.g. a peptide, that delivers the drug selectively to a target and/or to the cytosol of a cell. These compounds can be produced from compounds of the present invention according to well-known methods. Other examples of prodrugs are compounds, wherein the carboxylate in a compound of the present invention is for example converted into an alkyl-, aryl-, choline-, amino, acyloxymethylester, linolenoyl-ester.

Metabolites of compounds of the present invention are also within the scope of the present invention.

Where tautomerism, e.g., keto-enol tautomerism, of compounds of the present invention or their prodrugs may occur, the individual forms, e.g., the keto or the enol form, are claimed separately and together as mixtures in any ratio. The same applies for stereoisomers, e.g., enantiomers, cis/trans isomers, conformers and the like. If desired, isomers can be separated by methods well known in the art, e.g. by liquid chromatography. The same applies for enantiomers, e.g., by using chiral stationary phases. Additionally, enantiomers may be isolated by converting them into diastereomers, i.e., coupling with an enantiomerically pure auxiliary compound, subsequent separation of the resulting diastereomers and cleavage of the auxiliary residue. Alternatively, any enantiomer of a compound of the present invention may be obtained from stereoselective synthesis using optically pure starting materials

The compounds of the present invention can be in the form of a pharmaceutically acceptable salt or a solvate. The term “pharmaceutically acceptable salts” refers to salts prepared from pharmaceutically acceptable non-toxic bases or acids, including inorganic bases or acids and organic bases or acids. In cases where the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups can be present in salt form, and can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the present invention which contain one or more basic groups, i.e. groups which can be protonated, can be present in salt form, and can be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts can be obtained by customary methods which are known to a person skilled in the art, for example by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Furthermore, the present invention relates to pharmaceutical compositions comprising a compound of the present invention, or a prodrug compound thereof, or a pharmaceutically acceptable salt or solvate thereof as an active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

A pharmaceutical composition of the present invention may additionally comprise one or more other compounds as active ingredients, such as one or more additional compounds of the present invention, or a prodrug compound or other BTK inhibitors.

The pharmaceutical compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In one embodiment, said compounds and pharmaceutical composition, or pharmaceutically acceptable salt, prodrug or hydrate thereof, and a pharmaceutically acceptable carrier, are for the treatment of cancer.

The invention also relates to the use of compounds according to the invention for the preparation of a medicament for the treatment of cancer.

In practical use, the compounds of the present invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like. In the case of oral liquid preparations, any of the usual pharmaceutical media may be employed, such as, for example, suspensions, elixirs and solutions; or carriers such as starches, sugars, microcrystalline cellulose, diluents, granulating agents, lubricants, binders, disintegrating agents and the like. In the case of oral solid preparations the composition may take forms such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of the present invention may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of the present invention are administered orally.

The effective dosage of active ingredient employed may vary depending on the particular compound employed, the mode of administration, the condition being treated and the severity of the condition being treated. Such dosage may be ascertained readily by a person skilled in the art.

In certain aspects, the invention provides a capsule comprising the compound as described above. In certain embodiments, the capsule comprises about 20 mg to about 100 mg of the compound of the invention. In certain embodiments, the capsule comprises about 80 mg of the compound of the invention. In certain embodiments, the capsule comprises about 160 mg of the compound of the invention. In certain embodiments, the capsule comprises about 600 mg of the compound of the invention. In certain embodiments, the capsule comprises about 900 mg of the compound of the invention. In certain embodiments, the compound is N-[(1-acryloylpiperidin-4-yl)methyl]-5-(4-phenoxyphenyl)pyrimidine-4,6-diamine (A250) or 1-(4-(((6-amino-5-(4-phenoxyphenyl) pyrimidin-4-yl) amino) methyl)-4-fluoropiperidin-1-yl)prop-2-en-1-one (A225). In certain embodiments, the compound is N- [(1-acryloylpiperidin-4-yl)methyl]-5-(4-phenoxyphenyl)pyrimidine-4,6-diamine (A250). In certain embodiments, the compound is 1-(4-(((6-amino-5-(4-phenoxyphenyl)pyrimidin-4-yl)amino)methyl)-4-fluoropiperidin-1-yl) prop-2-en-1-one (A225).

In certain aspects, the invention is directed towards a method of treating a cancer as described herein, comprising the step of administering to a subject the capsule, as described above, once per day. In certain embodiments, the cancer is Relapsed/Refractory B Cell Malignancies. In certain embodiments, the cancer is selected from Non-Hodgkin's Lymphoma mantle cell lymphoma, and Non-Hodgkins's Lymphoma diffuse large b-cell lymphoma, including abc subtype.

The invention also relates to a set (kit) consisting of separate packs of
a) an effective amount of a compound according to the invention or a physiologically acceptable salt, solvate or prodrug thereof, and
b) an effective amount of a further medicament active ingredient.

The set comprises suitable containers, such as boxes, individual bottles, bags or ampoules. The set may, for example, comprise separate ampoules, each containing an effective amount of a compound according to the invention and/or pharmaceutically usable derivatives, solvates and stereoisomers thereof, including mixtures thereof in all ratios, and an effective amount of a further medicament active ingredient in dissolved or lyophilised form.

### EXAMPLES

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

Compound numbers utilized in the Examples below correspond to compound numbers set forth supra.

### Example 1

### Mosaic Spot Assay (CLL Patient Samples)

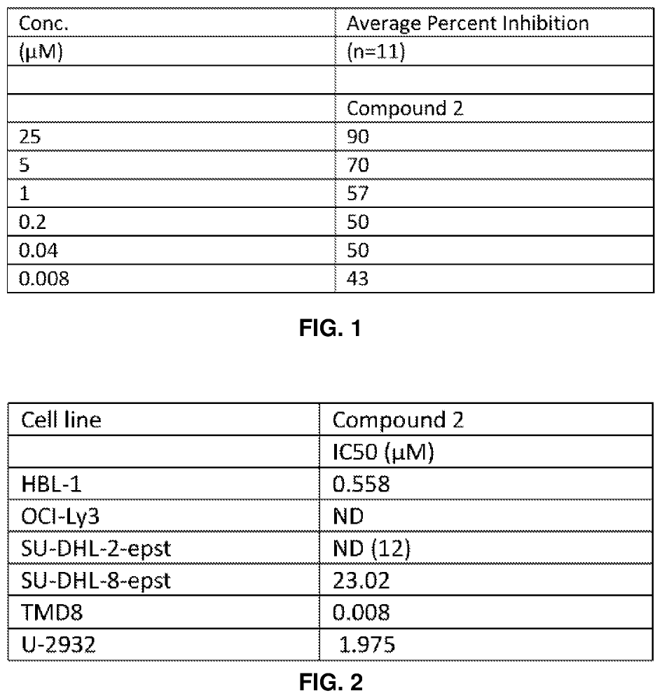

The Mosaic Spot Assay was conducted in accordance with Mosaic Laboratories' SOPs. The CLL blood and bone marrow mononuclear cells were evaluated for viability and cell count prior to plating in Mosaic Spot Media (X-Vivo 10 Media [Lonza, Fisher Scientific, Carlsbad, CA]+10% FBS [Life Technologies, Carlsbad, CA]) in a 96-well cytophobic plate in the presence of compounds and two controls: 1) a cytotoxic dose of cisplatin serving as a positive control; and 2) no drug treatment serving as a reference and negative control. Tumor cells were placed in a humidified 37° C. incubator with 5% CO2 for 4 days. At the end of the incubation, cells were removed and cytocentrifuged onto positive charged glass slides (Superfrost+, VWR, Radnor, PA). Slides were stained with Wright Giemsa in accordance with Mosaic Laboratories SOPs. Cell growth was measured by a hematopathologist. The results are provided in FIG. 1.

### Example 2

### MCL/ABC-DLBCL cell lines

Cell lines that were preserved in liquid nitrogen were thawed and expanded in growth media containing full serum. Once cells reached expected doubling times, screening began. Cells were seeded in growth media in black 384-well tissue culture treated plates. Cells were equilibrated in assay plates via centrifugation and placed in incubators (attached to the Dosing Modules) at 37° C. for twenty-four hours before treatment. At the time of treatment, a set of assay plates (which do not receive treatment) were collected and ATP levels were measured by adding ATPLite (Perkin Elmer). These Tzero (T₀) plates were read using ultra-sensitive luminescence on Envision plate readers. Assay plates were incubated with compound for seventy-two hours and were analyzed using ATPLite. All data points were collected via automated processes and were subject to quality control and analyzed using

Horizon's proprietary software. Assay plates were accepted if they passed the following quality control standards: relative raw values were consistent throughout the entire experiment, Z-factor scores were greater than 0.6 and untreated/vehicle controls behaved consistently on the plate.

Horizon utilized Growth Inhibition (GI) as a measure of cell growth. The GI percentages were calculated by applying the following test and equation:

$$If T < {V}_{0} : 100 * ( 1 - \frac{T - {V}_{0}}{{V}_{0}} ) \\ If T \geq {V}_{0} : 100 * ( 1 - \frac{T - {V}_{0}}{V - {V}_{0}} )$$

where T is the signal measure for a test article at seventy-two hours, V is the untreated/vehicle-treated control measure, and V₀ is the untreated/vehicle control measure at time zero (also colloquially referred as T₀ plates). This formula was derived from the Growth

Inhibition calculation used in the National Cancer Institute's NCI-60 high throughput screen.

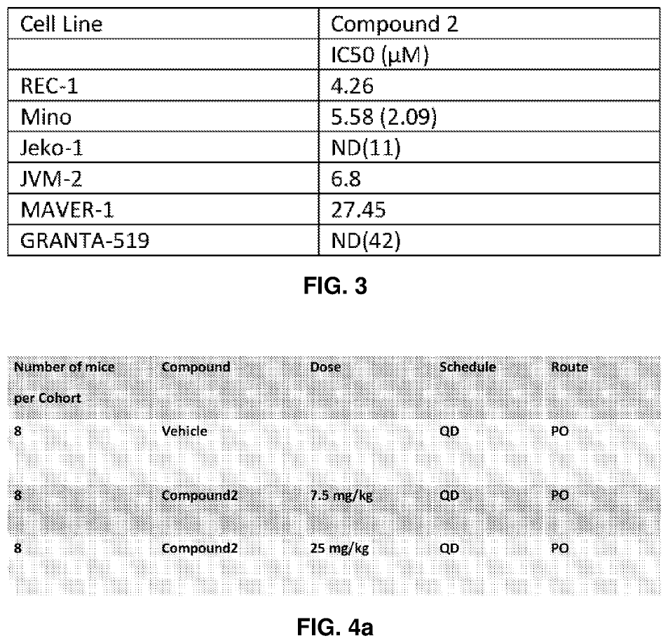

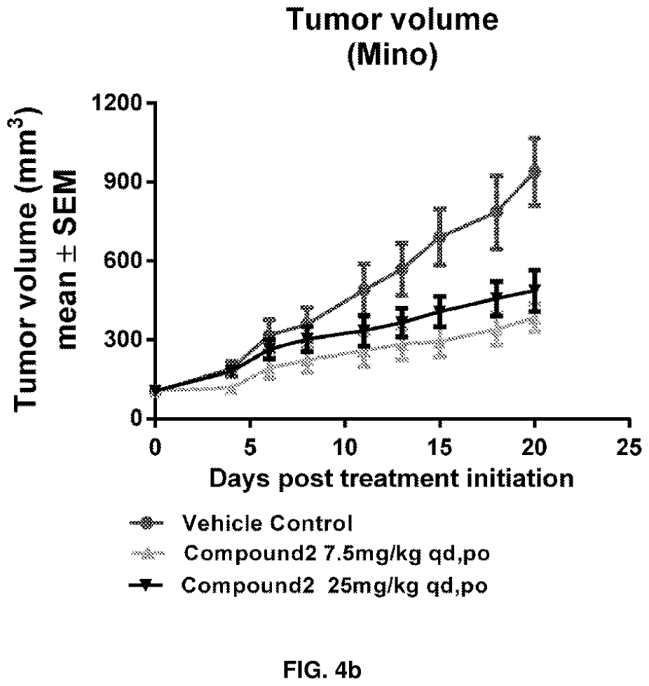

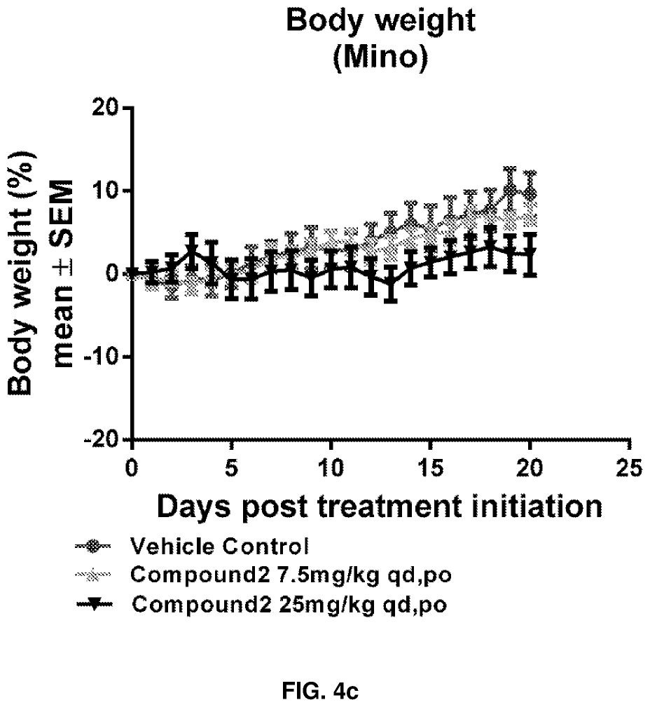

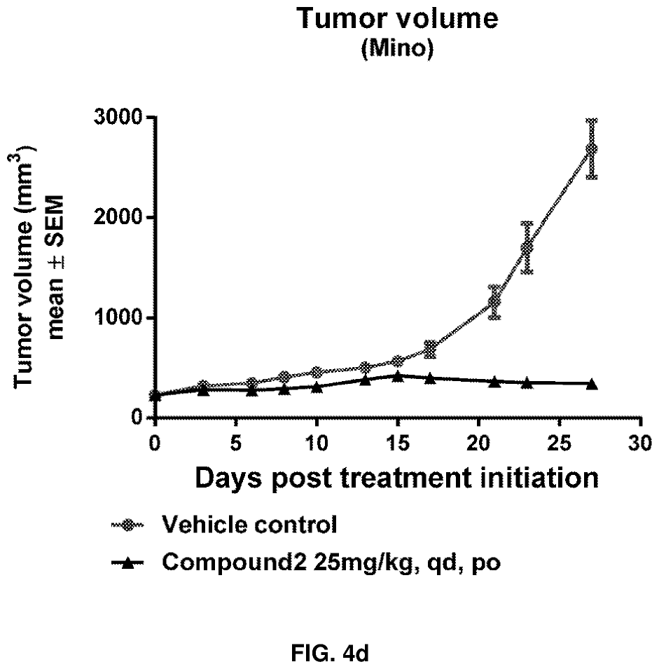

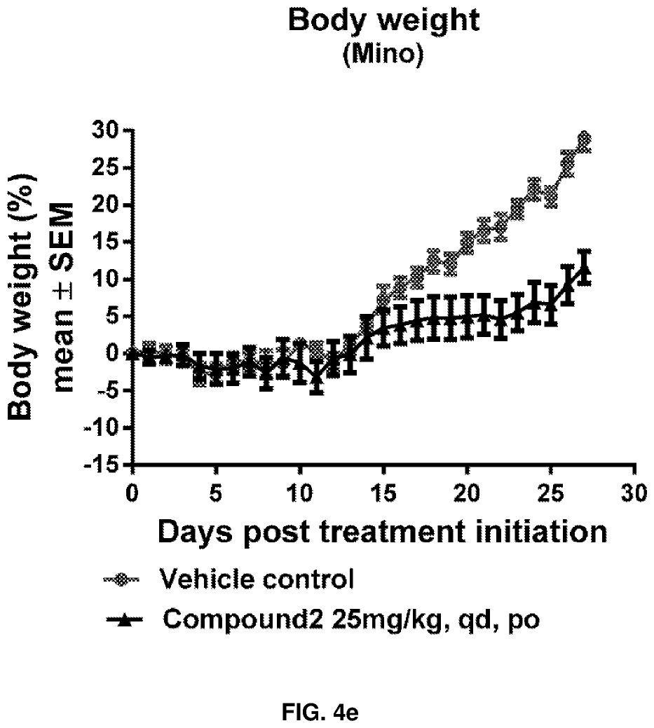

A GI reading of 0% represents no growth inhibition and occured in instances where the T reading at seventy-two hours were comparable to the V reading at the respective time period. A GI 100% represents complete growth inhibition (cytostasis) and in this case cells treated with compound for seventy-two hours had the same endpoint reading as To control cells. A GI of 200% represents complete death (cytoxicity) of all cells in the culture well and in this case the T reading at seventy-two hours was lower than the To control (values near or at zero). These GI calculations were used in all single agent and combination data analysis, and the results are found in FIGS. 2 and 3.

What is claimed is:

1. A method of treating acute myeloid leukemia in a human subject, comprising:
administering a therapeutically effective amount of a compound of the formula 1-(4-(((6-amino-5-(4-phenoxyphenyl)pyrimidin-4-yl)amino)methyl)-4-fluoropiperidin-1-yl)prop-2-en-1-one or a pharmaceutically acceptable salt thereof to the human subject.

2. The method of claim 1, wherein the compound of the formula 1-(4-(((6-amino-5-(4-phenoxyphenyl)pyrimidin-4-yl)amino)methyl)-4-fluoropiperidin-1-yl)prop-2-en-1-one is administered to the human subject.

3. The method of claim 1, wherein the pharmaceutically acceptable salt of the compound of the formula 1-(4-(((6-amino-5-(4-phenoxyphenyl) pyrimidin-4-yl)amino)methyl)-4-fluoropiperidin-1-yl) prop-2-en-1-one is administered to the human subject.

4. The method of claim 1, wherein the compound or the pharmaceutically acceptable salt thereof is administered to the human subject orally.

5. The method of claim 1, wherein the compound of the formula 1-(4-(((6-amino-5-(4-phenoxyphenyl)pyrimidin-4-yl)amino)methyl)-4-fluoropiperidin-1-yl)prop-2-en-1-one or the pharmaceutically acceptable salt thereof is administered as a pharmaceutical composition comprising the compound and at least one pharmaceutical carrier.

6. The method of claim 5, wherein the pharmaceutical composition comprises about 20 mg to about 100 mg of the compound.

7. The method of claim 6, wherein the pharmaceutical composition comprises 20 mg of the compound.

8. The method of claim 6, wherein the pharmaceutical composition comprises 50 mg of the compound.

9. The method of claim 6, wherein the pharmaceutical composition comprises 80 mg of the compound.

10. The method of claim 6, wherein the pharmaceutical composition comprises 100 mg of the compound.

11. The method of claim 5, wherein the pharmaceutical composition comprises about 160 mg of the compound.

12. The method of claim 5, wherein the pharmaceutical composition comprises about 600 mg of the compound.

13. The method of claim 5, wherein the pharmaceutical composition comprises about 900 mg of the compound.

14. The method of claim 5, wherein the pharmaceutical composition is a capsule containing the compound.

15. The method of claim 5, wherein the pharmaceutical composition is administered to the human subject orally.

16. The method of claim 5, wherein the pharmaceutical composition is administered twice daily.

17. The method of claim 1, wherein the therapeutically effective amount eliminates is an amount that eliminates binding to off-target polypeptides.

18. The method of claim 1, wherein about 7.5 mg/kg of the compound is administered orally once per day.

19. The method of claim 1, wherein about 25 mg/kg of the compound is administered orally once per day.

20. The method of claim 1, wherein the treatment achieves a normal level of cell proliferation.
